(19) United States
(12) Patent Application Publication
(10) Pub. No.: US 2021/0301329 A1
(43) Pub. Date: Sep. 30, 2021

(54) Single Cell Genetic Analysis

(71) Applicant: Cellecta, Inc., Mountain View, CA (US)

(72) Inventors: Alex Chenchik, Redwood City, CA (US); Russell Paul Darst, IV, San Bruno, CA (US); Lester Kobzik, San Carlos, CA (US); Mikhail Makhanov, San Francisco, CA (US); Donato Tedesco, Berkeley, CA (US)

(21) Appl. No.: 17/189,640

(22) Filed: Mar. 2, 2021

Related U.S. Application Data
(60) Provisional application No. 62/994,034, filed on Mar. 24, 2020.

(51) Int. Cl. C12Q 1/6834

(52) U.S. Cl. CPC C12Q 1/6834

(57) ABSTRACT

Single cell genetic analysis methods are provided. Aspects of the methods include: (a) producing a plurality of partitioned cell/barcoded bead complexes from a cellular sample and a plurality of distinct barcoded beads that include a plurality of barcoded reverse gene-specific primers; (b) hybridizing gene-specific template binding domains of the barcoded reverse gene-specific primers to template nucleic acids of the cells to produce primed template nucleic acids; and (c) subjecting the primed template nucleic acids to primer extension reaction conditions sufficient to produce barcoded nucleic acids, e.g., for subsequent amplification and analysis, such as by Next Generation Sequencing (NGS) protocols. Also provided are compositions that find use in practicing embodiments of the methods.

### CROSS-REFERENCE TO RELATED APPLICATION

Pursuant to 35 U.S.C. § 119(e), this application claims priority to U.S. Provisional Application Ser. No. 62/994,034 filed Mar. 24, 2020, the disclosure of which application is herein incorporated by reference.

### INTRODUCTION

Biomedicine has entered an era of advances at the cellular and molecular level. The goal of researchers and clinicians alike is to understand and then modify cell behavior through molecular techniques and tools. The methodologies for assessing cell biology at a molecular level are numerous. They include analyses of genomic DNA sequences, epigenetics, chromatin structure, messenger RNA (mRNA), non-protein-coding RNA, protein expression or modifications, and metabolites.

One area that has proven especially productive is the study of mRNA molecules (collectively termed the ‘transcriptome’), whose expression correlates well with important cellular features and with changes in cellular state. Transcriptomics was first applied to large pools of millions of cells, starting with hybridization-based microarrays, and later with next-generation sequencing (NGS) methods referred to as RNA-seq. RNA-seq on pooled cells has provided a vast amount of data that continues to spark discovery and innovation in biomedicine.

One limitation of the pooled approach is that the results represent an average of a large number of cells, often comprised of mixtures of cells differing from each other with respect to identity, phenotype and/or function. The nature of pooled cell studies does not allow detailed evaluation of the fundamental biological unit—the individual cell. This limitation has been addressed with the development of single cell genetic analysis methods, such as single cell DNA sequencing (scDNA-seq) or single cell RNA sequencing (scRNA-seq) approaches. scRNA-seq can identify and quantify RNA molecules in individual cells with high resolution and on a genome-wide scale. Indeed, use of scRNA-seq methods has increased rapidly. This reflects the recognition that biomedical researchers and clinicians can make important new discoveries using this powerful approach.

One major use of scRNA-seq has been to delineate transcriptional similarities and differences within a population of cells. For example, early studies revealed previously unappreciated levels of heterogeneity in embryonic and immune cells. Thus, the remarkable heterogeneity of seemingly identical cell populations remains a core reason for investigations using scRNA-seq.

Similarly, scRNA-seq can identify transcriptional differences between individual cells which allows identification of rare cell populations that would otherwise go undetected in analyses of pooled cells, such as malignant cancer cells within a tumor mass, or hyperresponsive immune cells within a seemingly homogeneous group. scRNA-seq is also ideal for examination of single cells where each cell is essentially phenotypically unique, such as the analysis of individual T lymphocytes expressing unique T-cell receptors, or neurons within the brain, scRNA-seq is also increasingly being used to trace lineage and developmental relationships between heterogeneous, yet related, cellular states in embryonal development, cancer, organ-specific epithelium differentiation and lymphocyte fate diversity.

Although variations and custom modifications abound in the published literature, a general workflow for scRNA-seq studies can be summarized as follows. The first step in conducting scRNA-seq is isolation of viable, single cells (or nuclei) from the experimental sample, e.g., cells grown in vitro, blood, tissue of interest. Current methods then rely on isolating partitioning of these single cells or nuclei thereof together with barcoded oligonucleotides attached to beads into physically separate compartments/partitions (e.g., microwells) or into individual droplets within microfluidic devices (e.g., as discussed in greater detail below). For single-cell analysis each compartment usually comprises one cell and one bead, where oligonucleotides attached to the bead have the same unique bead-specific (cell-specific) barcode. Next, isolated individual cells are lysed to release mRNA molecules, which then hybridize with barcoded oligo dT primers attached to or released from the bead. Next, the resultant oligo dT-primed mRNAs are converted to barcoded complementary DNA (cDNA) by a reverse transcriptase. Barcoded cDNAs derived from different cells are then mixed together and amplified for the follow-up expression analysis.

Depending on the scRNA-seq protocol, the reverse-transcription primers usually also have adaptor sequences for use in the amplification step, unique molecular identifiers (UMIs) to mark unequivocally a single mRNA molecule, as well as bead- or cell-specific barcode sequences to label the sequences coming from an individual cell. The tiny amounts of cDNA are then amplified by PCR-based methods. Then, amplified and barcoded cDNAs are sequenced by NGS, using library preparation, sequencing methods and genome-alignment tools similar to those used for bulk samples.

### SUMMARY

Single cell genetic analysis methods are provided. Aspects of the methods include: (a) producing a plurality of partitioned cell/barcoded bead complexes from a cellular sample and a plurality of distinct barcoded beads that include a plurality of barcoded reverse gene-specific primers; (b) hybridizing gene-specific template binding domains of the barcoded reverse gene-specific primers to template nucleic acids of the cells to produce primed template nucleic acids; and (c) subjecting the primed template nucleic acids to primer extension reaction conditions sufficient to produce barcoded nucleic acids, e.g., for subsequent amplification and analysis, such as by Next Generation Sequencing (NGS) protocols. Also provided are compositions that find use in practicing embodiments of the methods.

### BRIEF DESORPTION OF THE FIGURES

FIGS. 1A and 1B provide schematics of scRNA-seq protocols according to embodiments of the invention, including the steps of complex formation between cells and barcoded beads, partitioning of cell-barcoded bead complexes (in droplets), hybridization of barcoded gene-specific primers with target template (RNA), pooling together barcoded gene-specific primer-template hybrids, amplification of barcoded cDNAs by multiplex RT-PCR and analysis of amplified barcoded cDNAs by NGS.

FIG. 2 provides a schematic of a scRNA-seq protocol that employs a partitioning of cell-barcoded bead complexes in microwells, according to an embodiment of the invention.

FIG. 3 provides a schematic of a scRNA-seq protocol that includes a sorting step of cell-barcoded bead complexes, according to an embodiment of the invention.

FIG. 4 provides a schematic of a multiplex RT-PCR protocol based on sequential primer extension reactions from reverse and forward primers, followed by an amplification step using universal anchor primers, according to an embodiment of the invention.

FIG. 5 provides a schematic of a single-cell genetic screen using cells transduced with effector constructs, production and partitioning of cell-barcoded bead complexes, and generation and analysis of scRNA-seq data, according to an embodiment of the invention.

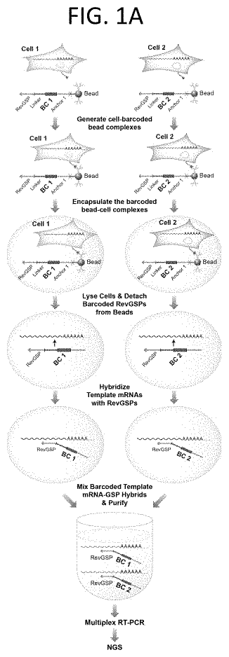

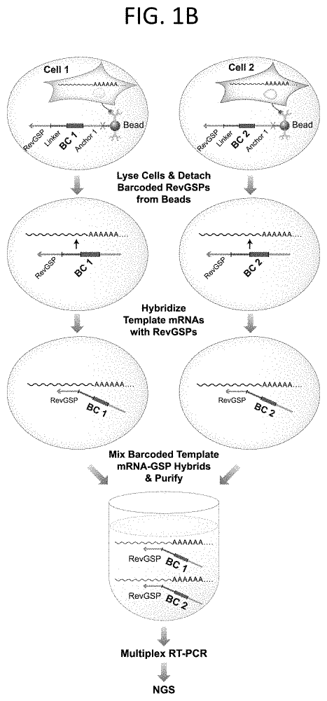

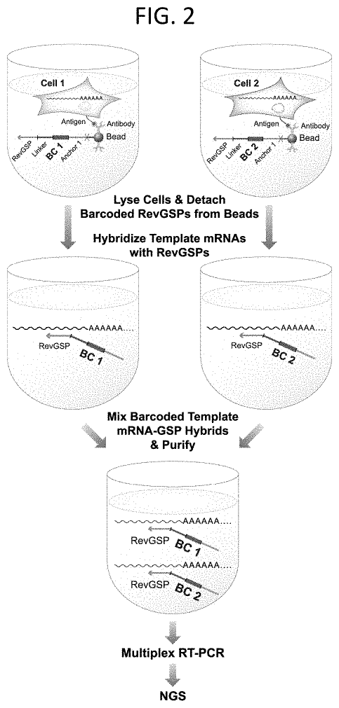

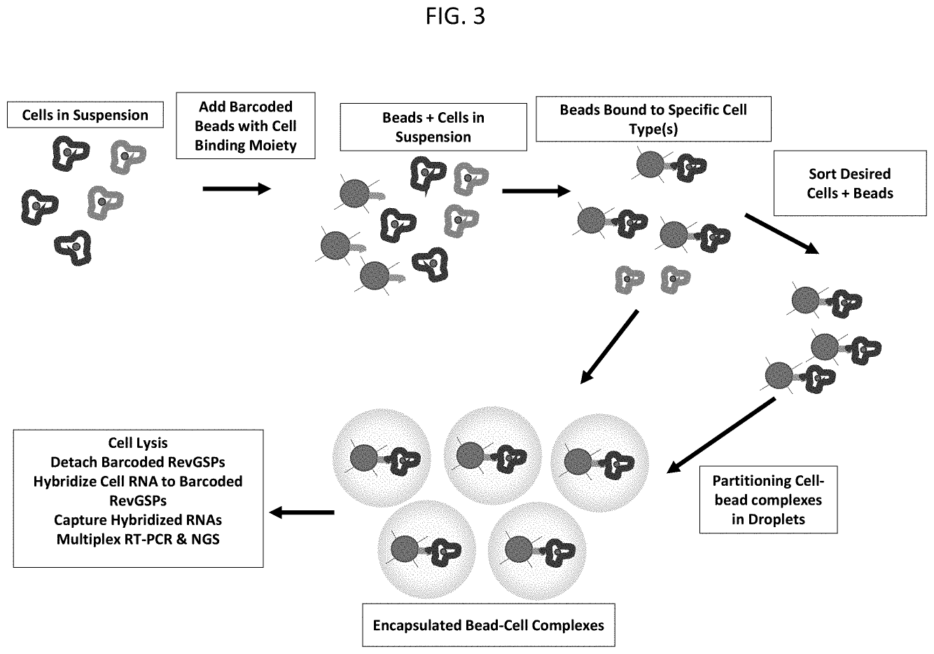

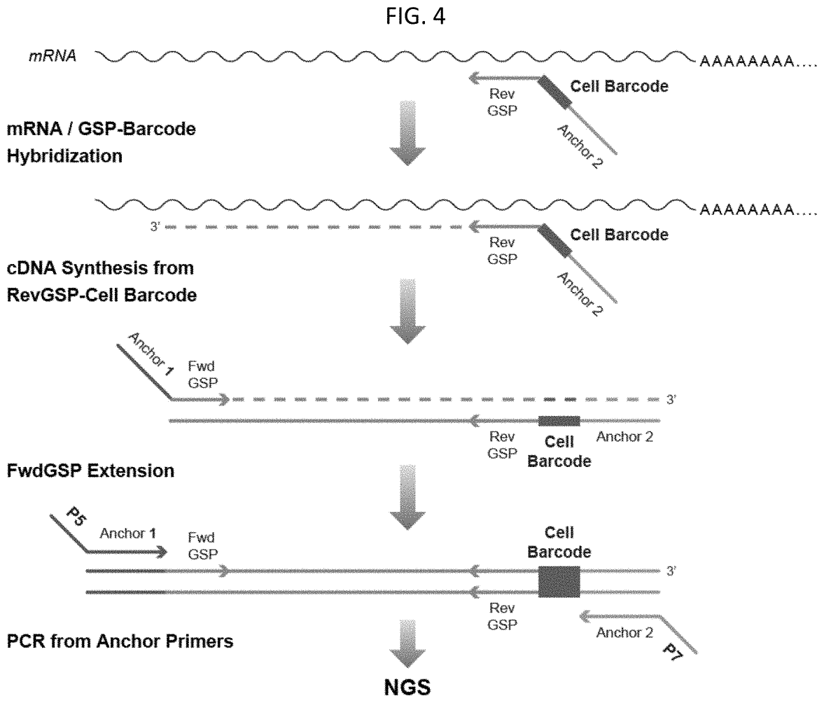

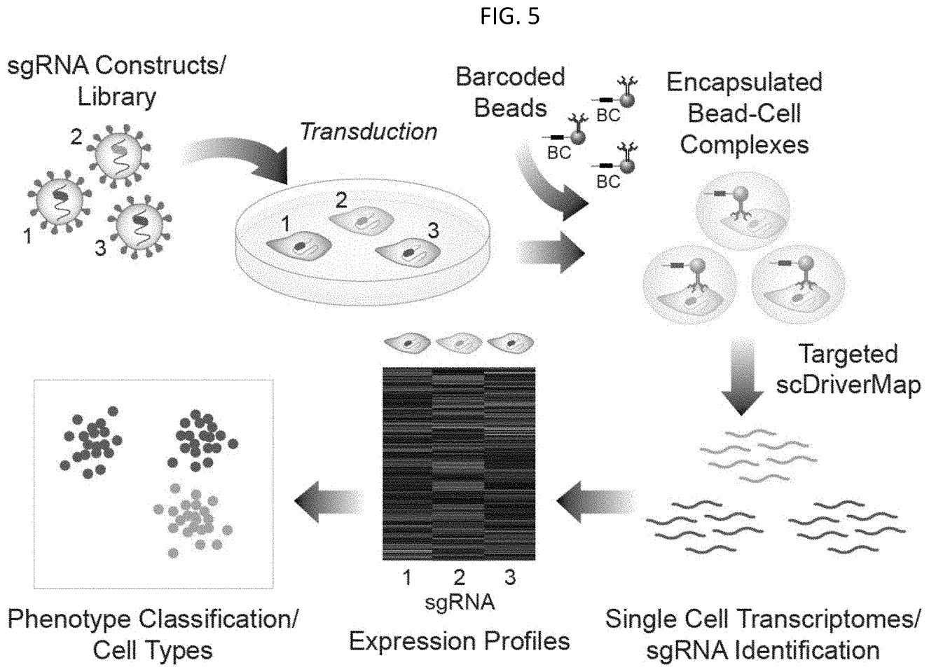

### DEFINITIONS

As used herein, the term “hybridization conditions” means conditions in which a primer, or other oligonucleotide, specifically hybridizes to a region of a target nucleic acid with which the primer or other oligonucleotide shares significant complementarity. Whether a primer specifically hybridizes to a target nucleic acid is determined by such factors as the degree of complementarity between the oligonucleotide and the target nucleic acid and the temperature at which the hybridization occurs, which may be informed by the melting temperature (TM) of the primer. The melting temperature refers to the temperature at which half of the primer-target nucleic acid duplexes remain hybridized and half of the duplexes dissociate into single strands. The Tm of a duplex may be experimentally determined or predicted using the following formula Tm=81.5+16.6(log 10[Na+])+0.41 (fraction G+C)−(60/N), where N is the chain length and [Na+] is less than 1 M. See Sambrook and Russell (2001; Molecular Cloning: A Laboratory Manual, 3rd ed., Cold Spring Harbor Press, Cold Spring Harbor N.Y., Ch. 10). Other more advanced models that depend on various parameters may also be used to predict Tm of primer/target duplexes depending on various hybridization conditions. Approaches for achieving specific nucleic acid hybridization may be found in, e.g., Tijssen, Laboratory Techniques in Biochemistry and Molecular Biology-Hybridization with Nucleic Acid Probes, part I, chapter 2, “Overview of principles of hybridization and the strategy of nucleic acid probe assays,” Elsevier (1993).

The terms “complementary” and “complementarity” as used herein refer to a nucleotide sequence that base-pairs by non-covalent bonds to all or a region of a target nucleic acid (e.g., a protein-coding region of the gene). As described in a more detail below, a barcoded oligonucleotide primer may be complementary to, and therefore may hybridize to, a target nucleic acid and therefore form a primed target nucleic acid hybrid. In the canonical Watson-Crick base pairing, adenine (A) forms a base pair with thymine (T), as does guanine (G) with cytosine (C) in DNA. In RNA, thymine is replaced by uracil (U). As such, A is complementary to T and G is complementary to C. In RNA, A is complementary to U and vice versa. Typically, “complementary” refers to a nucleotide sequence that is at least partially complementary. The term “complementary” may also encompass duplexes that are fully complementary such that every nucleotide in one strand is complementary to every nucleotide in the other strand in corresponding positions. In certain cases, a nucleotide sequence may be partially complementary to a target, in which not all nucleotides are complementary to every nucleotide in the target nucleic acid in all the corresponding positions. For example, a primer may be perfectly (i.e., 100%) complementary to the target nucleic acid, or the primer and the target nucleic acid may share some degree of complementarity which is less than perfect (e.g., 70%, 75%, 85%, 90%, 95%, 99%).

The percent identity of two nucleotide sequences can be determined by aligning the sequences for optimal comparison purposes (e.g., gaps can be introduced in the sequence of a first sequence for optimal alignment). The nucleotides at corresponding positions are then compared, and the percent identity between the two sequences is a function of the number of identical positions shared by the sequences (i.e., % identity=# of identical positions/total # of positions×100). When a position in one sequence is occupied by the same nucleotide as the corresponding position in the other sequence, then the molecules are identical at that position. A non-limiting example of such a mathematical algorithm is described in Karlin et al., Proc. Natl. Acad. Sci. USA 90:5873-5877 (1993). Such an algorithm is incorporated into the NBLAST and XBLAST programs (version 2.0) as described in Altschul et at, Nucleic Acids Res. 25:389-3402 (1997). When utilizing BLAST and Gapped BLAST programs, the default parameters of the respective programs (e.g., NBLAST) can be used. In one aspect, parameters for sequence comparison can be set at score=100, wordlength=12, or can be varied (e.g., wordlength=5 or wordlength=20).

A “domain” refers to a stretch or length of a nucleic acid made up of a plurality of nucleotides, where the stretch or length provides a defined function to the nucleic acid. Examples of domains include primer binding or anchor domains, hybridization (template-binding or gene-specific primer) domains, barcode domains (such as source/sample barcode domains), unique molecular identifier (UMI) domains, Next Generation Sequencing (NGS) adaptor domains, NGS indexing domains, etc. In some instances, the terms “domain” and “region” may be used interchangeably, where the length of a given domain may vary, in some instances the length ranges from 2 to 100 nucleotides (nt), such as 5 to 50 nt, e.g., 5 to 30 nt.

“Barcoded beads” (or barcoded oligonucleotide beads) are polymeric, hydrogel, glass, metal or composite particles with covalently or non-covalently attached barcoded oligonucleotides. In some embodiments, all oligonucleotides attached to a given bead have the same barcode domain (which barcode domain is specific for the bead but is different from that found in oligonucleotides of any other beads being used in a given assay), and the same anchor domain. Furthermore, the barcoded oligonucleotides may include a template-binding domains or gene-specific primer domains which could be a plurality of different sequences, e.g., gene-specific primer compositions complementary to the target nucleic acid sequences, e.g., as described in greater detail below.

By “primer extension product composition” is meant a nucleic acid composition that includes nucleic acids that are primer extension products. Primer extension products are deoxyribonucleic acids that include a primer domain at the 5′ end covalently bonded to a synthesized domain at the 3′ end, which synthesized domain is a domain of base residues added by a polymerase mediated extension reaction to the 3′ end of the primer domain. The synthesized domain is a sequence that is dictated by a template nucleic acid to which the primer domain is hybridized and formed primed template nucleic acid compositions during production of the primer extension product. Primer extension product compositions may be single-stranded or double stranded nucleic acids that include a template nucleic acid strand complementary to a primer extension product strand, e.g., as described above. The length of the primer extension products and/or double stranded nucleic acids that incorporate the same in the primer extension product compositions may vary, wherein in some instances the nucleic acids have a length ranging from 50 to 1000 nt, such as 60 to 400 nt and including 70 to 250 nt. The number of distinct nucleic acids that differ from each other by sequence in the primer extension product compositions produced via methods of the invention may also vary, ranging in some instances from 10 to 50,000, such as 100 to 20,000 and including 1,000 to 10,000, 10,000 to 20,000, 15,000 to 20,000 and 15,000 to 19,000.

“Barcode” is the domain in the oligonucleotide attached to bead which is specific to that individual bead. A given barcode domain may vary in length, and in some instances ranges from 6 to 100 nt, such as from 10-50 nt and including from 12-30 nt. Barcode domains may be synthesized by conventional combinatorial or split-pool synthesis protocols using bead-oligonucleotide conjugates, wherein an initial oligonucleotide (e.g., attached the beads) includes any common domain(s), such as primer binding or anchor domains. In a split-pool strategy, the plurality of bead-oligonucleotide conjugates is split into several separate compartments (e.g. 4) and unique nucleotides or stretches of nucleotides are attached to the 3′-ends of oligonucleotides, e.g., A in compartment 1, G in compartment 2, C in compartment 3 and T in compartment 4. In the next synthesis cycle, all the beads pooled together, split again into several compartments and the next barcode-specific sequence is added to the each subpool oligonucleotide. The split-pool synthesis usually continues until each bead carries a unique barcoded oligonucleotide specific for that bead. Synthesis of barcoded beads can be performed by conventional phosphoramidite chemistry or by enzymatic addition of barcoded sub-domains using any conventional protocol, e.g., based on ligation or primer extension reaction. In the combinatorial ligation or primer extension strategy, the barcode domain may be built from several sequential rounds of adding oligonucleotides comprising barcode subdomains of suitable length, e.g., 4-12 nucleotides. “Barcoded bead/cell complex” means a composition that includes at least one cell or component thereof (e.g., nucleus) and one barcoded bead, where the cell and bead may be attached to each other, e.g., via a specific binding pair interaction such as cellular binding moiety attached to the bead. In some embodiments, e.g., to study cell-to-cell interactions, the complex could include two or more cells and one bead. In another application the complex could be used with one cell attached to two beads each with unique barcode. Barcoded beads and cells could be attached to each other through covalent or non-covalent bonds. Covalent bonds could be formed by using cross-linking reagents. Non-covalent interaction between barcoded beads and cells can be achieved by attaching a cell interacting/binding moiety to the bead, where examples of cell binding moieties include antibodies, aptamers, lipid molecules, etc., which cell interacting moieties could interact with and bind to moieties present in the cell surface. Cell interacting moieties may be non-specific with respect to cell type (e.g., a lipid cell interacting moieties that interacts with a cell membrane or a moiety interacting with cell surface based on electrostatic, hydrophobic, etc., interactions), specific for a given cell type (e.g., an antibody recognizing cell-type specific antigen), or a combination of both. The cell/barcoded bead complex interaction may be sufficiently stable to allow for separation of complexes from each other, e.g., by FACS, dilution, binding to a surface, droplet partitioning, etc.

“Cellular sample” means a liquid composition of plurality of cells, e.g., eukaryotic cells, or components thereof, e.g., nuclei. A cellular sample may be obtained from a biological source, such as normal or diseased tissue, biological fluids (blood, saliva, lymphatic liquid, etc.), cell fractions or cells grown in vitro, ex vivo or in vivo. A cellular sample obtained from biological source could be used directly or treated with physical, biological or chemical entities (e.g., anti-cancer drugs) prior the use in the single cell assay. A cellular sample could also be fixed (e.g. with cross-linking reagent) prior to assay. In some instances, a cellular sample is a plurality of single cells isolated from a biological source by dissociation of multicellular structures or cell aggregates, e.g., using any convenient protocol. In some embodiments, a cell sample may include cellular structural components, such as nucleus, cytoplasm, mitochondria derived from single cell and having DNA or RNA component, etc. In some embodiments a cell sample includes two or more cells (e.g., organoids, cluster of cells) which are attached together based on natural cell-cell interactions necessary to perform a biological function (e.g., stroma-epithelial, immune-cancer, etc. cell-cell interaction). In some embodiment of the current invention, the cellular sample is made up of a plurality of cells genetically modified by delivering genetic effector constructs in target cells by conventional protocols, e.g., viral transduction. As disclosed U.S. Pat. Nos. 9,429565 and 10,196,634 (the disclosures of which are herein incorporated by reference), the effectors comprise a wide range of molecules including sgRNA, shRNA, aptamers, antisense RNA, microRNAs, peptide, native or modified proteins, etc., which effectors may be expressed in the target cells and change the cells' genotype and/or phenotype. The expression of effector molecules may change expression or regulation of target genes (e.g., drug targets), express modified version of target proteins (e.g., oncogenic mutated proteins), etc. The expression of effector molecules is a key technology for genetic screen and studying gene functions, e.g., discovery of novel drug targets for development of novel drugs. The effector constructs may also include clonal barcodes which allows for labeling each genetically modified cell and its progeny with cell-specific barcodes. Clonal barcodes, e.g., as described in the above patents, may be used for labeling both genomic DNA and expressed effector RNAs in individual cells, therefore providing additional (not bead derived) barcode for cell tracing. Clonal barcodes are further described in U.S. Pat. Nos. 9,429,565 and 10,196,634, the disclosures of which are herein incorporated by reference. Single cell analysis using protocols disclosed in the current invention with genetically modified cells allows one to link expression profile with effector molecules in each specific cellular clone.

### DETAILED DESCRIPTION

Single cell genetic analysis methods are provided. Aspects of the methods include: (a) producing a plurality of partitioned cell/barcoded bead complexes from a cellular sample and a plurality of distinct barcoded beads that include a plurality of barcoded reverse gene-specific primers; (b) hybridizing gene-specific template binding domains of the barcoded reverse gene-specific primers to template nucleic acids of the cells to produce primed template nucleic acids; and (c) subjecting the primed template nucleic acids to primer extension reaction conditions sufficient to produce barcoded nucleic acids, e.g., for subsequent amplification and analysis, such as by Next Generation Sequencing (NGS) protocols. Also provided are compositions that find use in practicing embodiments of the methods. The methods and compositions described herein find use in a variety of different applications, including single-cell expression profiling of RNAs and proteins, mutation, structural variation and epigenetic analysis in genomic DNA, gene function analysis, drug target, small molecule and biologics screening applications.

Before the present invention is described in greater details, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

While the apparatus and method has or will be described for the sake of grammatical fluidity with functional explanations, it is to be expressly understood that the claims, unless expressly formulated under 35 U.S.C. § 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 U.S.C. § 112 are to be accorded full statutory equivalents under 35 U.S.C. § 112.

In further describing various aspects of the invention, embodiments of various methods will be discussed first in greater detail, followed by a review of various applications in which the methods find use as well as kits that find use in various embodiments of the invention.

### Methods

As summarized above, methods of preparing barcoded nucleic acids are provided. Aspects of the methods include: (a) producing a plurality of partitioned cell/barcoded bead complexes from a cellular sample and a plurality of distinct barcoded beads that include a plurality of barcoded reverse gene-specific primers under conditions sufficient to; (b) hybridizing gene-specific template binding domains of barcoded reverse gene-specific primers to template nucleic acids of the cells to produce primed template nucleic acids; and (c) subjecting the primed template nucleic acids to primer extension reaction conditions sufficient to produce barcoded nucleic acids, e.g., for subsequent amplification and analysis, such as by Next Generation Sequencing (NGS) protocols, Each of these aspects is now described in greater detail.

### Production of Partitioned Cell/Barcoded Bead Complexes

Embodiments of the methods include producing a plurality of partitioned cell/barcoded bead complexes. By partitioned cell/barcoded bead complexes is meant that the cell/barcoded bead complexes, e.g., as described below, are separated from each by barrier, which may be liquid or solid, such that nucleic acids from one complex do not interact with nucleic acids from another complex of the plurality of partitioned complexes. In some instances, partitioning involves isolation of one cell (or component thereof, e.g., nucleus) with one barcoded bead in a physically separated compartment prior to lysis of the cell. One cell-one bead compartmentalization allows one to perform enzymatic reactions or physical interaction between the RNA or DNA templates present or released from single cell with barcoded oligonucleotides attached or released from beads. Furthermore, compartmentalization allows one to minimize the contamination between different compartments by the nucleic acids of other cells in an experimental sample or by barcoded oligonucleotides released from beads. Partitioning may be accomplished using any convenient protocol, as desired. In some instances, partitioning/compartmentalization is performed by one of two main approaches: droplet-based methods or physical isolation of one cell—one bead composition into microwell compartments and sealing of these microwells. Droplet-based platforms include, but are not limited to, for example, Chromium from 10× Genomics, ddSEQ from Bio-Rad Laboratories, InDrop from 1CellBio, and μEncapsulator or Nadia from Dolomite Bio/Blacktrace Holdings. The latter microwell approach includes, but is not limited to: commercial platforms such as the BD Rhapsody, the ICELL8 Single-Cell System (Takara) or custom protocols that rely on flow cytometric sorting or random deposition of single cells and barcoded beads or barcoded oligonucleotides into wells of microplates usually in two sequential steps.

As such, in some instances, the partitioned cell/bead complexes are present in microdroplets. For example, “water in on” microdroplets may be generated by mixing cells with barcoded beads and oil-surfactant composition using a wide range of known-in-art microfluidics designs, e.g., T-junction, step-out, co-flow, etc. The droplet generator device allows one to generate thousands of micron-sized aqueous compartments in oil-surfactant composition. In conventional protocol, cell and barcoded bead compositions are mixed in a limiting dilution prior to the droplet generation step. The combination of compartment size and limiting dilution of the cells and barcoded beads is used to generate compartments containing, on average based on double Poisson distribution, just single cell-single bead, single cell-no bead, single bead-no cell compositions with low percentage of compartments comprising more than one cell or more than one bead. Embodiments of the current invention, based on the generation of pre-formed one cell-one barcoded bead complexes prior to encapsulation stage allows for significantly improved encapsulation efficiency of single cell-single bead composition in droplets. Furthermore, the desired one cell-one barcoded bead complexes could be further enriched (e.g. by FACS) prior encapsulation step. The average size of a compartment in an water in oil droplet emulsion ranges from 50 microns in diameter to over 100 microns, depending on the specific microfluidics design, but usually diameter of droplets is at least 2-fold more than diameter of beads, which are usually in the range of 10-80 microns and cells (10-20 microns). Depending on the size of the droplets and volume of cell sample, up to 1×10⁶ individual encapsulated cell-barcoded bead compositions could be generated and assayed in the same container, e.g. tube, well or other suitable container. Protocols that may be employed include those that allow one to deliver individual cells with unique barcoded beads and reagents necessary for reverse transcription step into separate microdroplets. Such microdroplet technologies include the Chromium instrument (10× Genomics), the ddSeq instrument (Bio-Rad), etc. Microdroplets that include compositions as described above may also be generated and delivered to separate compartments or to oil (to form water-oil droplets) using other than microfluidics conventional technologies, e.g., FACS, ink-jet deposition, etc.

In other embodiments, the partitioned compositions are present or delivered to microwells of microplates with well sizes dimensioned to accommodate individual cells and barcoded beads, where the dimensions may be configured to accommodate on average no more than 2 cells or 2 beads, such as no more than 1 cell or 1 bead. Examples of such microwells are those found in the plates of the Rhapsody instrument (Becton, Dickinson and Company), the ICELL8 instrument (Takara Bio USA), etc. where such instruments employ plates having approximately 10,000-200,000 wells and a deposition protocol for individual cells and single beads. In other embodiments, the cells and beads could be delivered to microchambers using microfluidics technology using chips developed by Fluidigm Corporation.

The partitioned cell/barcoded bead complexes may include a single cell or component thereof (e.g., nucleus) and a single barcoded bead, or two or more cells (or components thereof) and a single barcoded bead, or a single cell (or component thereof) and two or more barcoded beads. Of interest in some embodiments are partitioned cell/barcoded bead complexes that include a single cell or single nucleus and a single barcoded bead. As such, barcoded beads may interact with a cell (or nuclei isolated from cells) population to produce partitioned cell/barcoded bead complexes made up of single cell-single bead pairs, or cell/barcoded bead complexes comprised of a single barcoded bead and two or more cells or a single cell bound to two or more barcoded beads. In some instances, single cell-single bead complexes are of interest since they provide the specific genetic analysis of a cell population at single cell resolution as identified by the barcoding sequences of the barcoded reverse primers. The generation of partitioned single cell/barcoded bead complexes with low percentage of multiple bead or multiple cell complexes may be achieved by optimizing the ratio between cells and barcoded beads, e.g., using an excess of beads from number of cells. In some instances, small numbers of complexes comprised of one cell with multiple beads or one bead with multiple cells may enter the analytical workflow. In the case of one cell binding multiple beads, the resultant genetic profile of the cell will be attributed to two or more cells. This is unlikely to skew results significantly, based on the low frequency of these events and the preservation of signature of the cell, albeit now divided by two or more bead-specific barcodes into two or more separate but similar profiles within the population of cells under study. For the case of one bead binding multiple cells, this may lead to confounding results e.g., in transcriptional analysis, since this may attribute incorrectly high levels of expression of certain genes to a single bead-specific barcode since the single bead's oligonucleotides will now be capturing the RNA from two or more cells. The magnitude of one-bead-multiple cell complexes can be assessed using cells genetically labeled (e.g., by viral transduction with barcoded genetic constructs) or labeled by an additional barcoded oligonucleotide (e.g. using cell hashing technology based on binding of barcoded oligonucleotides with cells). Furthermore, if the cells are labeled with unique genetic or oligonucleotide barcodes, the cell-bead complexes derived from two or more cells could be identified by analysis of extension products comprising more than one genetic/oligonucleotide barcode and single bead barcode. In some applications, e.g., those designed for analysis of cell-interaction, the binding of one bead to two or more cells is beneficial as such allows one to identify and profile the cells which are naturally close and interact with each other in vivo. One bead-two cell complexes may be isolated by FACS or other suitable technology from biological sample, e.g., tissue sample partially disintegrated to the level of 1-5 cell aggregates.

Barcoded beads employed in methods of the invention may vary. In some instances, the barcoded beads include a bead component having present on the surface thereof a plurality of distinct barcoded gene-specific reverse primers. The bead component can be made of a polymeric material (e.g., polystyrene, acrylamide, hydrogel, polymethylmethacrylate, etc.) but may be made of other materials as well (e.g., glass, metal, magnetic bead with iron core surrounded by polymeric shell, etc.). The beads can be non-modified or chemically modified at the surface (e.g., sulfated, amidated, carboxylated, etc.) to provide for binding to oligonucleotides or to use as a starting support for oligonucleotide synthesis. The size of the beads may vary, where in some instances the diameter of the beads ranges from 1 to 1,000 microns, such as 2 to 500 microns, including 3 to 200 microns, e.g., 10-80 microns, e.g., 20-40 microns. In some instances, the size of the bead is selected to correspond to the size of the cellular component of the cell/barded bead complexes to be produced in a given protocol. For example, where a given protocol is a single cell analysis protocol, the barcoded beads may have a diameter ranging from 10 to 80 microns. For analysis of cell clusters (e.g. organoids), the barcoded beads may have a diameter ranging from 40 to 200 microns. The shape of the beads may also vary, ranging from spherical structure to other shapes (e.g., cylinder, cube, irregular, etc.). The bead components may be non-porous or porous, e.g., where pores may be provided to impart a higher surface density of immobilized molecules. The beads may also be covered by a polymeric layer to increase the amount of attached barcoded oligonucleotides and increase conjugation efficiency of attached oligonucleotides.

The barcoded beads employed in methods of the invention include beads with a plurality of distinct barcoded gene-specific reverse primers attached thereto. While the number of distinct barcoded gene-specific reverse primers attached to any given bead may vary, in some instances the number of distinct barcoded gene-specific reverse primers attached to any given bead is 10⁴ or more and 10¹² or less, and in some instances the number ranges from 10⁵ to 10¹², such as 10⁶ to 10¹², including 10⁷ to 10¹¹ e.g., 10⁸ to 10¹⁰ distinct barcoded gene-specific reverse primers. In some instances, all distinct barcoded gene-specific reverse primers attached to a given bead have the same barcode domain, such that they share a common barcode domain. In other embodiments one bead could carry two or more barcode domains among the barcoded reverse primers attached thereto. In embodiments of the methods, within a given plurality of barcoded beads, the majority of, if not all of, the barcoded beads have different barcodes from each other. For example, if a given protocol is designed to profile 10,000 cells and uses 100,000 barcoded beads, the 100,000 barcodes attached to the 100,000 barcoded beads are significantly different from each other, such that at least 95%, such as 99% and including 99.9% of beads have different barcodes that are distinct from each other.

In embodiments, the barcoded reverse primers include a number of different domains, which domains may include a gene-specific template binding domain, a barcode domain and an anchor domain, wherein in some instances the order these domains from the 5′ end to the 3′ end is the anchor domain, the barcode domain and the template binding domain.

Anchor domains are domains that are employed in nucleic acid amplification steps of the methods, such as polymerase chain reaction (PCR), where anchor domains serve as primer binding sites for the primers employed in such amplification steps. Where the amplification employed is PCR, the anchor domains may also be referred to as PCR primer binding domains. The length of the anchor domains may vary, as desired. In some instances, anchor domains range in length from 10 to 50 nt, such as 15 to 30 nt, e.g., 18 to 28, including 18 to 26 nt. Where desired, the anchor domains may include PCR suppression sequences. PCR suppression sequences are sequences configured to suppress the formation of non-target DNA amplification products (e.g., primer dimers) during PCR amplification reactions, e.g., via the production of pan-like structures. Such sequences, when present, may vary in length, ranging in some instances from 5 to 25 nt, such as 7 to 21, including 7 to 20 nt. PCR suppression sequences of interest include, but are not limited to, those sequences described in U.S. Pat. No. 5,565,340; the disclosure of which is herein incorporated by reference. An example of forward and reverse anchor domains that include PCR suppression sequences are:

(SEQ ID NO: 01)
AGCACCGACCAGCAGACA
and
(SEQ ID NO: 02)
AGCACCGACCAGCACAGA.

Barcoded reverse primers also include a barcode domain. A barcode domain is a domain that denotes, i.e., indicates or provides, information about (such that it may be used to determine), the specific bead and therefore cell associated therewith in a given cell/barcoded bead complex, from which primed template nucleic acids are produced. Barcode domains include unique, specific sequences. While the length of a given barcode domain may vary, in some instances the length ranges from 6 to 60 nt, such as 8 to 40 nt, and including 12 to 20 nt.

Also present in the barcoded reverse primers are gene-specific template binding domains, where a given barcoded bead employed in embodiments of the invention includes a population of reverse gene-specific primer template binding domains. While the number of distinct gene-specific primer template binding domains (of differing sequence) in a given set that is associated with a given bead may vary, as desired, in some instances the number of distinct gene-specific domains in a given set is 10 or more, such as 20 or more, 30 or more, 40 or more, 50 or more, 60 or more, 70 or more, 80 or more, 90 or more, 100 or more, 125 or more, 250 or more, 500 or more, including 1000 or more, 2000 or more, 5000 or more, 8000 or more, 10,000 or more 15,000 or more, 18,000 or more and 20,000 or more. In some instances, the number of distinct gene-specific template binding domains that is present in a given set is 25,000 or less, such as 20,000 or less. As such, in some embodiments the number of gene-specific template binding domains of a bead that is employed in the methods ranges from 10 to 25,000, such as 50 to 20,000, including 1,000 to 10,000, e.g., 2,500 to 8,500, and 10,000 to 20,000, e.g., 15,000 to 19,000.

Gene-specific template binding domains employed in embodiments of the invention may be experimentally validated as suitable for use in a multiplex amplification assay. By “experimentally validated as suitable for use in a multiplex amplification assay” is meant that primers which include the domains of a given set have been experimentally tested in a multiplex amplification assay, such as described in United States Published Patent Application Nos. 20160376664 and 20180245164, the disclosures of which are herein incorporated by reference. To control efficiency and specificity of primer hybridization and the subject extension step, the length of the gene-specific domain of the gene-specific template binding domain may vary. In some instances, the length ranges from 10 to 120 nt, such as 15 to 75 nt, e.g., 16 to 50 nt, such as 18 to 45 nt, including 20 to 40 nt or 25 to 40 nt. The different gene-specific template binding domain may vary length in order to adjust the melting temperature of gene-specific template binding domains to a similar value. In some instances, the length the gene-specific template biding domain ranges from 25 to 80 nt, such as 30 to 70 nt, including 30 to 40 nt. Where desired, the gene-specific template binding domains may be GCA- and/or GCT-rich. By GCA- and/or GCT-rich is meant that the gene-specific primer domain has a substantial portion of G, C, A- and/or G, C, T nucleotides. While the number of such nucleotides in a gene-specific template binding domain may vary, in some instance the number of such sequences ranges from 75% to 100%, such as 85% to 100%. As the gene-specific template binding domains of such embodiments are GCA- and/or GCT-rich, the GC content of the gene-specific template binding domains is also high. While the GC content may vary, in some instances the GC content ranges from 40 to 90%, such as 45 to 85%, including 50 to 85%, e.g., 50 to 80%. Experimentally validated set of different gene-specific binding domains employed in the assay are usually have similar melting temperature (e.g. +/−5° C.) adjusted at design stage by length and GC composition as described above. Depending on the specific application for which the set is configured, the set of gene-specific template binding domains may be configured to target a wide range of mammalian genes, genetically modified genes or artificial or recombinant sequences (e.g. barcodes, genes, effector constructs, reporter and clonal barcode constructs, barcoded oligonucleotides used for cell labeling) introduced in the cells, and pathogenic genes from a wide range of pathogenic organisms, such as viruses, bacteria, fungi, etc. which could be present in the human or mammalian bodies. Of interest in certain applications are human or mammalian species commonly used as model organisms to study human diseases, such as mouse, rat, or monkey, and pathogenic organisms associated with human diseases. To be analyzed in accordance with embodiments of the invention, the targeted genes may be present in the mammalian cells or fluids. In some embodiments, the targeted genes are may be protein coding, or may express non-coding RNAs, micro RNAs, mitochondrial RNAs, regulatory RNAs, etc. In some instances, the set of genes selected is genome-wide, such that it covers all genes present in the genome of an organism. In other embodiments, the genes are selected from the genes that could be transcribed or expressed in the organism and present in the biological samples in the form of RNA. The genome-wide set of genes specific for human, model and pathogenic organisms is of special interest in some instances and may be used to develop a set of genome-wide targeted RNA expression assays based on the disclosed multiplex PCR assay. Genome-wide sets of gene-specific template binding domains may vary in number, and in some instances are configured to assay 18,000 or more, such as 20,000 or more and 25,000 or more, such as 30,000 or more genes. Additional sets of PCR primers may be configured based on a genome-wide set of genes from a wide range of viral, bacterial and eukaryotic pathogenic organisms. In another embodiment, the gene-specific template binding domains may be configured to produce primer extension products from a subset of specific genes selected from the genome-wide set of genes. Examples of sets of template gene-specific template binding domains and their use in single cell genetic analysis applications is disclosed in U.S. patent application Ser. Nos. 15/133,184 and 16/543,211, the disclosures of which sets of gene-specific reverse primers are incorporated herein by reference.

In addition to the above domains, the barcoded reverse primers of the barcoded beads may, where desired, include one or more additional domains. One type of additional domain that may be included is a unique molecular index (UMI) domain, UMI domains have sequences configured for labeling of each RNA molecule in a plurality of RNA molecules (and extended cDNA product) present in a hybridization mix with different molecule-specific indexes. UMI domains are stretches of random or semi-random nucleotides. While the lengths of UMI domains may vary, in some instances the length of a given UMI domain ranges from 8 to 20 nt, which in a given assay provides for complexity of different unique sequences of 10,000 or more different UMIs. In some instances, using at least 10,000 unique indexes is sufficient to label each template molecule present in one sample with a unique index, i.e., UMI. By analyzing the number of the indexes, e.g., via NGS, the number of each unique template molecules employed in multiplex PCR assay can be calculated. In some instances, when present the UMI domain may be combined with the barcode domain, e.g., where the UMI nucleotides are interspersed with the barcode nucleotides in a BUMI domain, e.g., as described in United States Patent Application Publication No. US20150072344, the disclosure of which is herein incorporated by reference.

In addition, barcoded reverse primers may include one or more linker domains. Linker domains are domains that link other domains together, e.g., barcode and gene-specific template binding domains. While the length of a given linker domain may vary, in some instances the length ranges from 5 to 30 nt, such as 10 to 25 nt, including 12 to 20 nt. There are no special requirements for nucleotide composition or sequence of the linker domain, but in some instances the linker domain is selected with GC-content in the range 50% to 80% without significant secondary structure within the domain or with other domains present in the oligonucleotide.

The barcoded nucleic acids, e.g., as described above (which may also be referred to barcoded reverse gene-specific primers, may be attached to the beads by non-covalent or covalent bonds. In some instances, the barcoded reverse gene-specific primers are covalently attached to the beads, e.g., through a suitable linker. While any convenient linker may be employed, in some instances the linker is a cleavable linker, such as a photocleavable linker, a chemically cleavable linker, a thermosensitive linker and the like, which cleavable linkers allow for the release of barcoded oligonucleotides or barcoded extended DNA fragments from beads when desired. Such linkers include labile moieties, such as light labile moieties, chemical/enzymatic labile moieties, thermal-labile moieties etc., where examples of such moieties are disclosed Published United States Patent Application Publication No. US 2019-0112648 A1; the disclosure of which moieties and linkers including the same is herein incorporated by reference. Examples of cleavable linkers that may be employed include, but are not limited to, thermal-labile linkers, enzymatically-labile linkers, light-labile linkers, etc. In some instances, the linker is a thermal labile linker that includes a thermally-labile blocking moiety. A thermally-labile blocking moiety is a moiety that may be cleaved when the temperature is raised above a certain threshold value to release barcoded primer from bead. While the threshold value may vary, in some instances the threshold value is 60° C. or higher, such as 75° C. or higher, including 90° C. or higher. Examples of thermally labile moieties that may be employed in accordance with the invention include, but are not limited to, those described in U.S. Pat. Nos. 8,133,669 and 8,361,753; the disclosures of which are herein incorporated by reference. In some instances, the thermally labile blocking moiety is a 3′ blocking moiety, such as but not limited to: O-phenoxyacetyl; O-methoxyacetyl; O-acetyl; O-(p-toluene)sulfonate; O-phosphate; O-nitrate; O-[4-methoxy]-tetrahydrothiopyranyl; O-tetrahydrothiopyranyl; O-[5-methyl]-tetrahydrofuranyl; O-[2-methyl,4-methoxy]-tetrahydropyranyl; O-[5-methyl]-tetrahydropyranyl; and O-tetrahydrothiofuranyl.

In some instances, the linker is an enzymatically-labile linker. An enzymatically-labile linker includes a moiety that may be cleaved by exposing the linker to a suitable enzyme that cleaves the moiety. Examples of enzymatically-labile moieties of interest include those having a linkage group cleavable by a hydrolase enzyme. Examples of hydrolase enzymes of interest include, but are not limited to: esterases, phosphatases, peptidases, penicillin amidases, glycosidases and phosphorylases, kinases, etc. Hydrolase susceptible linkages and hydrolase enzymes are further described in U.S. Patent Application Publication No. 20050164182 and U.S. Pat. No. 7,078,499; the disclosures of which are herein incorporated by reference.

In some instances, the linker is a chemically-labile linker that includes a chemically-labile moiety. A chemically-labile is a moiety that may be cleaved by exposing the linker to a chemical agent that cleaves the moiety. The chemically-labile moiety may be reactive with the functional group of a chemical agent (e.g., an azido-containing modifiable group that is reactive with an alkynyl-containing reagent or a phosphine reagent, or vice versa, or a disulfide that is reactive with a reducing agent such as tris(2-carboxyethyl)phosphine (TCEP) or DTT). A variety of functional group chemistries and chemical agent stimuli suitable for modifying them may be utilized in the subject methods. Functional group chemistries and chemical agents of interest include, but are not limited to, click chemistry groups and reagents (e.g., as described by Sharpless et al., (2001), “Click Chemistry: Diverse Chemical Function from a Few Good Reactions”, Angewandte Chemie International Edition 40 (11): 2004-2021), Staudinger ligation groups and reagents (e.g., as described by Bertozzi et al., (2000), “Cell Surface Engineering by a Modified Staudinger Reaction”, Science 287 (5460): 2007), and other bioconjugation groups and reagents (e.g., as described by Hermanson, Bioconjugate Techniques, Second Edition, Academic Press, 2008). In certain embodiments, the chemically-labile blocking moiety includes a functional group selected from an azido, a phosphine (e.g., a triaryl phosphine or a trialkyl phosphine or mixtures thereof), a dithiol, an active ester, an alkynyl, a protected amino, a protected hydroxy, a protected thiol, a hydrazine, and a disulfide.

In some instances, the cleavable linker is a light-labile linker that includes a light-labile moiety, which is a moiety that may be cleaved by exposing the linker to light at a wavelength that cleaves the moiety from the linker. Examples of light-labile moieties of interest include cleavable by light of a certain wavelength that cleaves a photocleavable group in the linkage group. Any convenient photocleavable groups may find use. Cleavable groups and linkers may include photocleavable groups comprising covalent bonds that break upon exposure to light of a certain wavelength. Suitable photocleavable groups and linkers for use in the subject MCIP's include ortho-nitrobenzyl-based linkers, phenacyl linkers, alkoxybenzoin linkers, chromium arene complex linkers, NpSSMpact linkers and pivaloylglycol linkers, as described in Guillier et al. (Chem. Rev. 2000 1000:2091-2157). For example, a 1-(2-nitrophenyl)ethyl-based photocleavable linker (Ambergen) can be efficiently cleaved using near-UV light, e.g., achieving >90% yield in 5-10 minutes using a 365 nm peak lamp at 1-5 mW/cm2. In some embodiments, the modifiable group is a photocleavable group such as a nitro-aryl group, e.g., a nitro-indole group or a nitro-benzyl group, including but not limited to: 2-nitroveratryloxycarbonyl, a-carboxy-2-nitrobenzyl, 1-(2-nitrophenyl)ethyl, 1-(4,5-dimethoxy-2-nitrophenyl)ethyl and 5-carboxymethoxy-2-nitrobenzyl. Nitro-indole groups of interest include, e.g., a 3-nitro-indole, a 4-nitro indole, a 5-nitro indole, a 6-nitro-indole or a 7-nitro-indole group, where the indole ring may be further substituted at any suitable position, e.g., with a methyl group or a halo group (e.g., a bromo or chloro), e.g., at the 3-, 5- or 7-position. In certain embodiments, the nitro-aryl group is a 7-nitro indolyl group. In certain instances, the 7-nitro indolyl group is further substituted with a substituent that increases the photoactivity of the group, e.g., substituted with a bromo at the 5-position. Any convenient photochemistry of nitroaryl groups may be adapted for use. In certain embodiments, the linker includes a photocleavable group, such as a nitro-benzyl protecting group or a nitro-indolyl group.

In a given plurality of barcoded beads, one or more domains of the barcoded reverse gene-specific primers attached to different beads of the plurality may be identical or common among the barcoded beads. For example, the barcoded reverse primers of a given plurality may include the same or common anchor domain, which domain may be employed for binding to universal PCR primers and for follow-up amplification of barcoded extended DNA fragments. Other domains that may be common among the barcoded oligonucleotides include linker domains, sample index domains, etc. Furthermore, among a given plurality of barcoded beads, the beads may include common gene-specific template binding domains. For example, the same plurality of distinct gene-specific template binding domains may be associated with bead of the plurality of barcoded beads.

For the purpose of capturing (binding) single cells of interest, in some instances the barcoded beads also include, in addition to the barcoded reverse gene-specific primers, a moiety capable of binding to a target cell of interest from cell sample, i.e., a cellular binding moiety. When present, the cellular binding moiety may vary, and may be a moiety capable of specific binding to cell or a structural component thereof. Examples of cellular binding moieties of interest include, but are not limited to: lipids, e.g., which bind to the lipid layer of cell membrane, aptamers, and proteinaceous specific binding members, e.g., antibodies or specific binding fragments thereof, which bind to a specific antigen on cell surface or nucleus surface. The cellular binding moiety may be bound directly to bead surface or bind (covalently or non-covalently) indirectly to oligonucleotides attached to beads, e.g., such that is bound to the bead surface of an oligonucleotide linker. In some instances, specific antibodies are coupled to an oligonucleotide and incubated with the beads carrying a complementary docking oligonucleotide, creating beads capable of directed binding to the surface of cells expressing the antigen(s) recognized by the antibodies docked to the beads via the coupled oligonucleotide sequence. Specific cell binding moiety domains of interest include, but are not limited to, antibody binding agents, proteins, peptides, haptens, nucleic acids, aptamers, lipids, etc. The term “antibody binding agent” as used herein includes polyclonal or monoclonal antibodies or fragments that are sufficient to bind to an analyte of interest. The antibody fragments can be, for example, monomeric Fab fragments, monomeric Fab′ fragments, or dimeric F(ab)′2 fragments. Also within the scope of the term “antibody binding agent” are molecules produced by antibody engineering, such as single-chain antibody molecules (scFv) or humanized or chimeric antibodies produced from monoclonal antibodies by replacement of the constant regions of the heavy and light chains to produce chimeric antibodies or replacement of both the constant regions and the framework portions of the variable regions to produce humanized antibodies. The marker of the cell of interest may be any convenient marker, such as a cell surface protein or structure having an epitope to which the specific binding domain may specifically bind. In such instances, the bead linked sample barcoded reverse primers may include one or more additional domains of interest, such as bead identifying domains (bead barcodes), antibody identifying domains (antibody barcodes), etc.

In one embodiment, the antibodies used can be one or both of a pair of antibodies selected for universal binding of a variety of human cells (e.g., anti-beta-2-microglobulin, anti-CD298). In other embodiments, antibodies specific for cell populations of interest can be used to limit binding of beads to specific cells, (e.g., anti-CD14 for blood monocytes). In some embodiments, several bead sets wherein each set includes an antibody for a specific cell type may be combined and used in the disclosed assay together. For these multiplex cell typing applications, the oligonucleotides attached to antibody (or barcoded oligonucleotides attached to beads) could comprise the antibody-specific barcode domain which will allow to incorporate these antibody-specific barcode In barcoded DNA extension products.

In other embodiments, the cellular binding moiety capable of mediating binding to specific types or cells in general can be used to prepare beads for specific binding of cells for subsequent genetic analysis. These cell binding moieties include, but are not limited to: lipids (e.g., as described in McGinnis et al., “MULTI-seq: sample multiplexing for single-cell RNA sequencing using lipid-tagged indices,” Nat Methods. (2019);16(7):619-26); lectins (etc., as described in Christiansen et al., “Identification of the major lectin-binding surface proteins of human neutrophils and alveolar macrophages,” Blood. (1988)71:1624-32); avidin (e.g., as described in Crupi et al., “Cell surface biotinylation of receptor tyrosine kinases to investigate intracellular trafficking,” Methods Mol Biol. (2015)1233:91-102); aptamers (e.g., as described in Zumrut et al., “Ligand-Guided Selection of Target-Specific Aptamers: A Screening Technology for Identifying Specific Aptamers Against Cell-Surface Proteins,” Nucl Acid Ther. (2016) 26:190-6); or other ligands for cell surface receptors or structures.

In one embodiment, barcoded beads with attached cell-specific binding moieties (e.g. antibodies) are incubated with suspensions containing the cells of interest, which could comprise all the cells within the suspension or a subset thereof. After binding, any resultant cell/barcoded bead complexes (e.g., made up of a single cell and single bead such as described above) may be isolated by flow cytometric sorting or used directly in the disclosed assay. Flow sorting allows one to employ various parameters to sort only specific cell population(s), e.g., antigen-specific cell fraction (e.g., CD45 cells) or sorting based on exclusion of fluorescent dyes to only sort live cell-bead complexes and exclude dead-cell-bead complexes.

As summarized above, methods of include partitioning cells of a cellular sample with a plurality of distinct barcoded beads comprising barcoded reverse gene-specific primers under conditions sufficient to produce a plurality of partitioned cell/barcoded bead complexes, e.g., as described above. Cellular samples may be derived from a variety of sources including but not limited to e.g., a cellular tissue, a biopsy, a blood sample, a cell culture, etc. Additionally, cellular samples may be derived from specific organs, tissues, tumors, neoplasms, or the like. Furthermore, cells from any population can be the source of a cellular sample used in the subject methods, such as a population of prokaryotic or eukaryotic single celled organisms including bacteria or yeast.

### Production of Primed Template Nucleic Acids

As reviewed above, following production of a plurality of partitioned cell-barcoded bead complexes, the methods then include producing primed template nucleic acids by hybridizing gene-specific template binding domains of barcoded reverse gene primers to template nucleic acids of the cells of the cell/barcoded bead complexes to produce primed template nucleic acids. Depending on a given protocol, the template nucleic acids of the primed template nucleic acids may vary. Essentially any nucleic acid template may find use in the subject methods, including e.g., RNA template nucleic acid and DNA template nucleic acids. RNA template nucleic acids may vary and may include e.g., messenger RNA (mRNA) templates, non-coding RNA, micro RNA, synthetic RNA delivered to cells, e.g. though transfection, RNA expressed from recombinant constructs (e.g. effector constructs) delivered to the cells using a wide range of delivery vectors, and the like. In addition, various types of DNA templates may be employed, including but not limited to e.g., genomic DNA templates, mtDNA templates, synthetic DNA templates, recombinant DNA templates, etc.

According to certain embodiments, the template nucleic acids are template ribonucleic acids (template RNA). Template RNAs may be any type of natural or/and artificial RNA or their combination present in cell sample. Natural RNA (or sub-type thereof) including, but not limited to, a messenger RNA (mRNA), a microRNA (miRNA), a transacting small interfering RNA (ta-siRNA), a natural small interfering RNA (nat-siRNA), a small nucleolar RNA (snoRNA), a small nuclear RNA (snRNA), a long non-coding RNA (lncRNA), a non-coding RNA (ncRNA), a transfer-messenger RNA (tmRNA), a precursor messenger RNA (pre-mRNA), a small Cajal body-specific RNA (scaRNA), a piwi-interacting RNA (piRNA), a small temporal RNA (stRNA), a signal recognition RNA, a telomere RNA, or any combination of RNA types thereof or subtypes thereof. Examples of artificial RNA which is usually delivered by synthetic or expressed from effector genetic constructs in a cell sample include, but are not limited to, a short hairpin RNA (shRNA), an endonuclease-prepared siRNA (esiRNA), a micro RNA, a small interfering RNA (siRNA), a single guide RNA (sgRNA), ribozyme, RNA encoding natural and genetically modified peptides, aptamers, proteins, clonal barcodes, UMI, genetic construct specific barcode (e.g., barcoded transcriptional reporter construct), regulatory RNA which could affect biological processes in target cell (e.g., as described in U.S. Pat. Nos. 9,429,565 and 10,196,634 (the disclosures of which are herein incorporated by reference), etc.

According to certain embodiments, the template nucleic acids are template deoxyribonucleic acids (template DNA). A template DNA may be any type of natural or genetically engineered DNA of interest to a practitioner of the subject methods, including but not limited to genomic DNA or fragments thereof, complementary DNA (or “cDNA”, synthesized from any RNA or DNA of interest), recombinant DNA (e.g., plasmid DNA), or the like. In some embodiments, the template nucleic acids are synthetic oligonucleotides delivered or bind to cells though cell binding moiety. Synthetic barcoded oligonucleotides conjugates with antibodies, lipids and other cell binding moieties are commonly used for labeling different cell sub-pools with different barcodes for single-cell analysis in the technologies, like Cell Hashing, etc.

To provide for access of the barcoded reverse primers of the barcoded beads to nucleic acids in the cells, the cell/barcoded bead complexes may be subjected to cell lysis/treatment/denaturation conditions which initiate interaction between cellular nucleic acids, e.g., mRNAs, and barcoded reverse primers. Where desired, chemical agents may be employed to lyse cells to allow release of nucleic acids (e.g., RNA). In other embodiments, in the cell lysis/hybridization step the cells or cell components (e.g. nuclei) are treated under mild-lysis conditions which do not lyse the cells but form holes in cellular/nuclear membrane which are necessary to initiate interaction between cellular nucleic acids and barcoded oligonucleotides. The cell lysis/hybridization step may be initiated by changing media surrounding cells with cell lysis solution using any convenient lysis composition, such as a cell lysis buffer solution containing denaturing agents (e.g., guanidium thiocyanate, urea, etc.), detergents (SDS, triton X100, NP40, etc.), hybridization accelerators (salt, polyethylene glycol, etc.), additives (EDTA, proteinase K, nuclease inhibitors, DTT, etc.) and the like. Any suitable lysis method may be employed. In some instances, a mild lysis procedure can advantageously be used to prevent the release of nuclear chromatin, thereby avoiding genomic contamination of a cDNA library, and to minimize degradation of mRNA. For example, heating cells at 60-70° C. for 2 minutes in the presence of Tween-20 is sufficient to lyse the cells while resulting in no detectable genomic contamination from nuclear chromatin. Alternatively, cells can be heated to 65° C. for 10 minutes in water (Esumi et al., Neurosci Res 60(4):439-51 (2008)); or 70° C. for 90 seconds in PCR buffer II (Applied Biosystems) supplemented with 0.5% NP-40 (Kurimoto et al., Nucleic Acids Res 34(5):e42 (2006)); or lysis can be achieved with a protease such as Proteinase K or by the use of chaotropic salts such as guanidine isothiocyanate (U.S. Patent application Publication No. 2007/0281313). Additional mild-lysis conditions, which do not destroy but permeabilize the cellular membrane, like treatment with methanol, detergents (Triton X-100, Tween-20, etc.), chaotropic salts (guanidium thiocyanate) in the presence of inorganic salts (sodium chloride) may be employed to initiate hybridization between barcoded reverse primers and cellular RNAs.

Treatment of cell/barcoded bead complexes under lysis conditions causes release of or otherwise makes accessible the nucleic acids (e.g., mRNA) from the cells of the cell/barcoded bead complexes. This release allows binding of RNA molecules to the barcoded reverse gene-specific primers provided by barcoded bead of the complex. In one embodiment, the barcoded reverse gene-specific primers are released from the barcoded bead by cleavage, such as by exposure to light (e.g., UV light) to activate cleavage of a photosensitive linker, causing release of the barcoded reverse gene-specific primers to allow greater interaction and hybridization with their complementary partners within the pool of cellular RNA now available for binding (e.g., after cell lysis by physical/chemical means). In another embodiment, the barcoded reverse gene-specific primers are not detached from the beads and hybridize to RNA molecules on the surface of the barcoded beads. In some embodiments, the hybridization step includes treatment of DNA and in some instances of RNA to make it more accessible to hybridization with the barcoded reverse gene-specific primers. These treatments may include any of a number of protocols, including fragmentation of DNA (e.g., ultrasound), treatment with enzymes (e.g., Proteinase K), heat denaturation (e.g., 95 C for 1 min), and the like. In some instances, the lysis and hybridization step is one step as lysis buffer composition may include the components which are necessary for hybridization step. The hybridization conditions (temperature, buffer compositions, time) may be optimized in order to provide high efficiency and specific interaction but minimize the non-specific interactions between the gene-specific template-binding domains and cellular nucleic acids.

As a result of hybridization, a plurality of primed template nucleic acids are produced for each cell/barcoded bead complex, which plurality of primed template nucleic acids is made up of hybridized nucleic acids comprising a template nucleic acid, e.g., mRNA or genomic DNA fragment, hybridized to a barcoded reverse gene-specific primer. The number of different primed template nucleic acids which differ from each other at least in terms of the template nucleic acid sequence may vary, where in some instances the number of distinct primed template nucleic acids in the plurality of primed template nucleic acids ranges from 1 to 200,000, 10 to 25,000, such as 100 to 20,000 and including 1,000 to 10,000, 10,000 to 20,000, 15,000 to 20,000 and 15,000 to 19,000. In embodiments, the different primed template nucleic acids have different barcoded reverse primers hybridized thereto since the barcoded reverse primers are gene-specific barcoded reverse primers.

Following production of the primed template nucleic acids from the disparate cell/barcoded bead complexes, the primed template nucleic acids from one or more different cell/barcode bead complexes may be combined or pooled for further processing. In such pooled compositions, each plurality of primed template nucleic acids derived from single cell/barcoded bead complex of the pooled composition will have a distinct barcode domain, such that the barcode domain of a first plurality of primed template nucleic acids of the composition will have a sequence that differs from every other barcode domain of every other plurality of primed template nucleic acids in the pooled composition. In a given pooled composition, each barcode domain has a sequence that is significantly different from that of any other barcode domain in the pooled composition, with a difference of at least 1 nucleotide, such as 2 nucleotides and including 3 or more nucleotide differences in the whole set of barcodes employed in the assay. In this way each plurality of the pooled composition will have a distinct identifying barcode domain. The number of different barcode domains in such pooled compositions is the same as the number of different pluralities in the pooled composition, where the number represents the number of different samples that is employed to make the pooled composition. The number of different barcodes present in a given pooled composition depends on number of samples being analyzed in a given assay. In some instances, the number ranges from 10 to 10,000,000, such as 10 to1,000,000, such as 100 to 1,000,000, such as 100 to 100,000, and including 1,000 to 100,000, such as 1,000 to 10,000. For example, currently for analysis of single-cell samples, the number of barcodes may be 10,000,000 or more, but for analysis of clinical samples the number of barcodes may not exceed 100,000.

Where desired, hybridization complexes of template and primer, i.e., primed template nucleic acids, may be purified, e.g., via separation from excess of non-bound primers, e.g., by nuclease treatment or/and binding to solid support, e.g., such as beads, e.g., as described below. In this way, excess of primers, such as gene-specific primers, may be removed in order to achieve a high specificity of primer extension reaction from the target template sequences. In some embodiments, prior to subjection to primer extension reaction conditions, e.g., as described in greater detail below, the plurality of primed template nucleic acids are combined together and purified from other constituents that may be present in the reaction mixture, such as non-bound barcoded reverse primers, non-hybridized nucleic acids, proteins, reverse transcriptase inhibitors, and the like. The purification of primed template nucleic acids may be achieved using any convenient protocol, e.g., by binding to a matrix, via fractionation based on size, charge, solubility, precipitation, etc. In one embodiment, the primed template nucleic acids are purified from non-primed nucleic acids, e.g., by using oligo dT-magnetic beads, followed by centrifugation or magnet binding steps and washing steps. In another embodiment, the primed template nucleic acids are separated from other components in the reaction mixture by contacting the mixture with a matrix (e.g., AMPure XP magnetic beads, glass particles (Qiagen), Silica particles (Thermo-Fisher), anion exchange resin (Qiagen), etc.) which specifically binds RNA or/and DNA molecules under optimized conditions but does not bind barcoded reverse primers. Some other protocols that may be employed include centrifugation, chromatography, precipitation, phase separation, etc. As a result of purification step, the plurality of prime template nucleic acids derived from different cells are purified from other components of the reaction mixture, e.g., non-bound oligonucleotides and other cellular components. The resultant purified primed template nucleic acids may be combined or pooled together, e.g, in a small volume of buffer, for subsequent primer extension to produce barcoded nucleic acids, e.g., as described below.

### Production of Barcoded Nucleic Acids

In methods of the invention, following production of the plurality of primed template nucleic acids, e.g., as described above, the primed template nucleic acids are subjected to primer extension reaction conditions sufficient to produce barcoded nucleic acids. The barcoded nucleic acids produced in this step include at least first strand DNA/cDNA flanked at one end, i.e., the 5′ end, with, among other optional domains, a reverse primer domain, a barcode domain and anchor domain, which domains have been provided to the barcoded nucleic acid by a barcoded reverse primer of a barcoded bead.

As reviewed above, in producing barcoded nucleic acids, primed template nucleic acids, e.g., as described above, are subjected to primer extension reaction conditions sufficient to produce the barcoded nucleic acids. By “primer extension reaction conditions” is meant reaction conditions that permit polymerase-mediated extension of a 3′ end of a nucleic acid strand, e.g., a barcoded reverse primer, hybridized to a template nucleic acid. Achieving suitable reaction conditions may include selecting reaction mixture components, concentrations thereof, and a reaction temperature to create an environment in which the polymerase is active and the specific primed nucleic acid may be extended.

In producing the primer extension reaction mixture, the primed template nucleic acids may be combined with a number of additional reagents (e.g., to increase specificity, uniformity, yield, etc. of extension products), which may vary as desired. A variety of polymerases may be employed when practicing the subject methods. Reference to a particular polymerase, such as those exemplified below, will be understood to include functional variants thereof unless indicated otherwise. Examples of useful polymerases include DNA polymerases, e.g., where the template nucleic acid is DNA. In some instances, DNA polymerases of interest include, but are not limited to: thermostable DNA polymerases, such as may be obtained from a variety of bacterial species and genetically modified to improve their performance, including Thermus aquaticus (Taq), Thermus thermophilus (Tth), Thermus filiformis, Thermus flavus, Thermococcus literalis, and Pyrococcus furiosus (Pfu) or modified and mutated versions of these DNA polymerases (e.g. Phusion DNA polymerase, Q5 DNA polymerase, etc.). Alternatively, where the target template nucleic acid composition is made up of RNA, the polymerase may be a reverse transcriptase (RT), where examples of reverse transcriptases include natural and genetically modified versions of Moloney Murine Leukemia Virus reverse transcriptase (MMLV RT), SuperScript II, SuperScript III, Maxima reverse transcriptase (Thermo-Fsher), SMARTScribe™ reverse transcriptase (Takara), AMV reverse transcriptase, Bombyx mori reverse transcriptase (e.g., Bombyx mori R2 non-LTR element reverse transcriptase), etc. In one embodiment, the enzymes with DNA polymerase activity are designed for hot-start primer extension reaction, e.g., used as a complex with specific antibody or chemical compound which blocks enzymatic activity at low temperature but fully releases the activity at reaction conditions. For example, in some instances a hot-start reverse transcriptase composition, e.g. complex between MMLV RT and Therma-Stop RT reagent (Thermagenix) or complex between MMLV RT and antibody is employed.

Primer extension reaction mixtures also include dNTPs. In certain aspects, each of the four naturally-occurring dNTPs (dATP, dGTP, dCTP and dTTP) are added to the reaction mixture. For example, dATP, dGTP, dCTP and dTTP may be added to the reaction mixture such that the final concentration of each dNTP is from 0.05 to 10 mM, such as from 0.1 to 2 mM, including 0.2 to 1 mM. According to one embodiment, at least one type of nucleotide added to the reaction mixture is a non-naturally occurring nucleotide, e.g., a modified nucleotide having a binding or other moiety (e.g., a fluorescent moiety) attached thereto, a nucleotide analog, or any other type of non-naturally occurring nucleotide that finds use in the subject methods or a downstream application of interest.

In addition to the primed template nucleic acids, the polymerase, and dNTPs, the reaction mixture may include buffer components that establish an appropriate pH, salt concentration (e.g., KCl concentration), metal cofactor concentration (e.g., Mg²⁺ or Mn²⁺ concentration), and the like, for the extension reaction and template switching to occur. Other components may be included, such as one or more nuclease inhibitors (e.g., an RNase inhibitor and/or a DNase inhibitor), one or more additives for facilitating amplification/replication of GC rich sequences (e.g., GC-Melt™ reagent (Takara Bio USA (Mountain View, Calif.)), betaine, single-stranded binding proteins (e.g., T4 Gene 32, cold shock protein A (CspA), recA protein, and/or the like) DMSO, ethylene glycol, 1,2-propanediol, or combinations thereof), one or more molecular crowding agents (e.g., polyethylene glycol, or the like), one or more enzyme-stabilizing components (e.g., DTT present at a final concentration ranging from 1 to 10 mM (e.g., 5 mM)), and/or any other reaction mixture components useful for facilitating polymerase-mediated extension reactions.

The primer extension reaction mixture can have a pH suitable for the primer extension reaction. In certain embodiments, the pH of the reaction mixture ranges from 5 to 9, such as from 7 to 9, including from 8 to 9, e.g., 8 to 8.5. In some instances, the reaction mixture includes a pH adjusting agent. pH adjusting agents of interest include, but are not limited to, sodium hydroxide, hydrochloric acid, phosphoric acid buffer solution, citric acid buffer solution, and the like. For example, the pH of the reaction mixture can be adjusted to the desired range by adding an appropriate amount of the pH adjusting agent.

The temperature range suitable for production of the product nucleic acid may vary according to factors such as the thermal stability of particular polymerase employed, the melting temperatures of any primers employed, etc. According to one embodiment, the primer extension reaction conditions include bringing the reaction mixture to a temperature ranging from 4° C. to 72° C., such as from 16° C. to 70° C., e.g., 37° C. to 65° C., such 30° C. as 55° C. to 65° C. The temperature of the reaction mixture may be maintained for a sufficient period of time for polymerase mediated, template directed primer extension to occur. While the period of time may vary, in some instances the period of time ranges from 5 to 60 minutes, such as 15 to 45 minutes, e.g., 30 minutes.

In some embodiments, the primer extension reaction conditions using RNA template may incorporate a template switching oligonucleotide, e.g., with sample-specific barcode domain, UMI and anchor domain. Template switch is described in U.S. Pat. Nos. 5,962,271 and 5,962,272, as well as Published PCT application Publication No. WO2015/027135; the disclosures of which are herein incorporated by reference. Where desired, the template switching oligonucleotide may be employed to introduce one or more domains at the 3′ end of the cDNA, such as but not limited to, an anchor domain, an adaptor domain or portion thereof, sample barcode domain, UMI domain, etc., e.g., as described in United States Published Patent Application Nos. 20150111789 and 20150203906, the disclosures of which are herein incorporated by reference. Template switching oligonucleotides may be employed in protocols where forward primers are not used, as desired. In some embodiments, the extended barcoded nucleic acids can be too short in order to design forward primers, like in the case of analysis of effector molecules sgRNA, shRNA, etc., small RNAs or in the cases wherein reverse primers are too close to the 5′ end of template nucleic acid. In other embodiments, target template nucleic acids could have polymorphic or mutated sequences downstream of reverse primers which prevent straightforward design of forward primers. Examples of such sequences are template nucleic acids for T-cell and B-cell receptors, wherein variable and leader domains of these genes are highly polymorphic and not well characterized, and highly mutated genes, like p53, NFkB, K-RAS, etc., in cancer samples, etc. For polymorphic, mutated or short transcripts the extended barcoded nucleic acids could incorporate template switching oligonucleotide at the 5′-end of RNA template. In another embodiment, the template switching oligonucleotide could be incorporated at the 3′-end of partially extended barcoded nucleic acids. As a result, the set of partially extended barcoded nucleic acids will start from the same reverse primers at the 5′-ends but generate overlapping set of extended products at the 3′-ends. Using overlapping sets of extended products allows one to read and reconstruct by alignment longer amplified products, e.g., up 700-800 nt or longer at next-generation sequencing step currently limited to approximately 300 nt (Illumina platform). The conditions for generation of truncated extension products terminated at the 3′-end with template switching oligonucleotide is well-known in art and include using unbalanced nucleotide composition, higher concentration of template switching oligonucleotide, modified buffer composition with additives like betaine, polyethylene glycol, etc. In another embodiment, the RNA template could be partially digested at any step of the protocol before reverse transcription to generate overlapping templates. The protocols for fragmentation of RNA template are well-known in art and include degradation of RNA with metals (e.g., Mg²⁺) at elevated temperature (e.g., 60° C.-95° C.), ribonucleases, etc. As a result of partial RNA fragmentation, barcoded extended product will incorporate template-switching oligonucleotide at the different positions at the 3′-end defined by the 5′-end of RNA fragments. Template switch protocols are further described in: Hagemann-Jensen et al., “Single-cell RNA counting at allele and isoform resolution using Smart-seq3,” Nat. Biotech. (June 2020) 38: 708-714; Ohtsubo et al., “Optimization of single strand DNA incorporation reaction by Moloney murine leukaemia virus reverse transcriptase,” DNA Research (2018) 25: 477-487; Ohtsubo et al., “Compounds that enhance the tailing activity of Moloney murine leukemia virus reverse transcriptase,” Scientific Reports (2017) 6: 6250; Kapteyn et al., “Incorporation of non-natural nucleotides into template-switching oligonucleotides reduces background and improves cDNA synthesis from very small RNA samples,” BMC Genomics (2010) 11:413; and Wulf, “Non-templated addition and template switching by Moloney murine leukemia virus (MMLV)-based reverse transcriptases co-occur and compete with each other,” J. Biol. Chem. (2019) 48: 18220-18231.

The resultant barcoded nucleic acid may, where desired, be contacted with a one or more forward primers, e.g., to introduce one or more desired domains to the end of the barcoded nucleic acid, where such domains may vary. In some instances, one or more forward primers is employed in an additional primer extension reaction to introduce a second anchor domain at the end of the barcoded nucleic acid that is opposite the end that includes the first anchor domain, e.g., to produce “sample-barcoded anchor-domain-flanked deoxyribonucleic acid (DNA) fragments”, by which is meant a DNA which is derived from genomic DNA or RNA templates and includes an anchor domain on each side of a gene-specific domain. In these instances, the forward primer(s) may vary. In some instances, a single forward primer is employed, where the primer includes a template binding domain that binds all or a desired conservative sequence/portion of the primer extension products from the first strand synthesis, e.g., where the template binding domain binds to a common sequence provided by a template switching oligonucleotide employed in first strand synthesis. In other embodiments, the universal forward primer binding domain is present in universal adaptor attached to the barcoded nucleic acid extension product. The universal adaptor could be attached to barcoded nucleic acid extension product by a wide range of known in art technologies, including ligation (Illumina True-Seq RNA-seq protocol), transposition (Illumina Tagmentation with Tn5 transoposon), etc. Alternatively, a plurality of different forward primers may be employed, such as a collection for forward gene-specific primers, e.g., that include a common anchor domain 5′ of a unique gene-specific domain. While the number of distinct primers in a given set may vary, as desired, in some instances the number of primers in a given set is 10 or more, such as 20 or more, 30 or more, 40 or more. 50 or more, 60 or more, 70 or more, 80 or more, 90 or more, 100 or more, 125 or more, 250 or more, 500 or more, including 1000 or more. 200 or more, 5000 or more, 8000 or more, 10,000 or more 15,000 or more, 18,000 or more and 20,000 or more. In some instances, the number of gene-specific primers that is present in the set is 25,000 or less, such as 20,000 or less. As such, in some embodiments the number of gene-specific primers in the set that is employed in the methods ranges from 10 to 25,000, such as 50 to 20,000, including 1,000 to 10,000, e.g., 2,500 to 8,500, and 10,000 to 20,000, e.g., 15,000 to 19,000. Gene-specific reverse primers include gene-specific domains, where these gene-specific domains may be experimentally validated as suitable for use in a multiplex amplification assay. By “experimentally validated as suitable for use in a multiplex amplification assay” is meant that primers for each target gene in a given set has been experimentally tested in a multiplex amplification assay, such as described in United States Published Patent Application Nos. 20160376664 and 20180245164, the disclosures of which are herein incorporated by reference. To control efficiency and specificity of primer hybridization and the subject extension step, the length of the gene-specific domain of the gene-specific primer may vary. In some instances, the length ranges from 10 to 120 nt, such as 15 to 75 nt, e.g., 16 to 50 nt, such as 18 to 40 nt, including 20 to 30 nt or 25 to 40 nt. The gene-specific domain primer may vary length. In some instances, the length of the gene-specific domain in the forward primers ranges from 16 to 40 nt, such as 18 to 30 nt. As the gene-specific forward primers may include additional domains, e.g., anchor domains, etc., in some embodiments the primers in length from 18 to 150 nt, such as 20 to 100 nt, including 20 to 75 nt, such as from 20 to 60 nt, including from 20 to 35 nt. Where desired, the gene-specific forward primers may be GCA- and/or GCT-rich. By GCA- and/or GCT-rich is meant that the gene-specific primer domain has a substantial portion of G, C, A- and/or G, C, T nucleotides. While the number of such nucleotides in a gene-specific primer domain may vary, in some instance the number of such sequences ranges from 75% to 100%, such as 85% to 100%. As the gene-specific primer domains of such embodiments are GCA- and/or GCT-rich, the GC content of the gene-specific primer domains is also high. While the GC content may vary, in some instances the GC content ranges from 40 to 90%, such as 45 to 85%, including 50 to 85%, e.g., 50 to 80%. Depending on the specific application for which the set is configured, the set of gene-specific primers may be configured to target a wide range of mammalian genes, genetically modified genes or artificial or recombinant sequences (e.g. barcodes, genes, effector constructs, synthetic oligonucleotides) introduced in the cells, and pathogenic genes from a wide range of pathogenic organisms, such as viruses, bacteria, fungi, etc. which could be present in the human or mammalian bodies. Of interest in certain applications are human, mammalian species commonly used as model organisms to study human diseases, such as mouse, rat, or monkey, and pathogenic organisms associated with human diseases. To be analyzed in accordance with embodiments of the invention, the targeted genes may be present in the mammalian cells or biological fluids, e.g. exosomes, circulating tumor DNA, etc. In some embodiments, the targeted genes are may be protein coding, or may express non-coding RNAs, micro RNAs, mitochondrial RNAs, regulatory RNAs, etc. In some instances, the set of genes selected is genome-wide, such that it covers all genes present in the genome of an organism. In other embodiments, the genes are selected from the genes that could be transcribed or expressed in the organism and present in the biological samples in the form of RNA. The genome-wide set of genes specific for human, model and pathogenic organisms is of special interest in some instances and may be used to develop a set of genome-wide targeted RNA expression assays based on the disclosed multiplex PCR assay. Genome-wide sets of primers may vary in number, and in some instances are configured to assay 18,000 or more, such as 20,000 or more and 25,000 or more, such as 30,000 or more genes. Additional sets of PCR primers may be configured based on a genome-wide set of genes from a wide range of viral, bacterial and eukaryotic pathogenic organisms. In another embodiment, the gene-specific primers may be configured to produce primer extension products from a subset of specific genes selected from the genome-wide set of genes. Examples of sets of gene-specific primers and their use in single cell genetic analysis applications is disclosed in U.S. patent application Ser. Nos. 15/133,184 and 16/543,211, the disclosures of which sets of gene-specific primers are incorporated herein by reference.

In the sample-barcoded anchor-domain-flanked deoxyribonucleic acid (DNA) fragments produced by embodiments of the invention, the gene-specific domain is one or several DNA fragments derived from one gene encoded by genomic DNA or RNA template. Where gene-specific primers are employed, e.g., as described above, the gene-specific domain may be specific sequence flanked from one or both sides with specific sequences of forward and reverse gene-specific primers. In one embodiment, the gene-specific domain is flanked from both sides by gene-specific primer sequences. In another embodiment, the gene-specific domain may correspond to the 3′-end sequence of an mRNA and be flanked from 5″-end by oligo dT sequences and from the other end by gene-specific primer or by anchor domain sequences which is non-specifically attached to an arbitrary gene sequence upstream of 3′-mRNA end (e.g., through ligation of anchor adaptor using transposase). A non-specific anchor domain may be also attached to the 3′-end of full-length or partially extended barcoded templates using e.g., template switching technology to provide a gene-specific domain flanked by one anchor domain at the 5′-end of mRNA molecule and gene-specific primer sequence or another non-specific to the sequence anchor domain.

As the gene-specific domain is flanked by anchor domains in these embodiments, the DNA fragments prepared by methods of the invention include a first anchor domain located at a first end of the DNA fragment and a second anchor domain located at a second end of the DNA. By gene-specific domain is meant a region of the dsDNA fragment the includes a sequence found in template target nucleic acid, such as a template target mRNA or DNA. While the length of the gene-specific domain may vary, in some instances the gene domain ranges in length from 20 to 1,000 nt, such as 50 to 500 nt, including as 60 to 300 nt.

In addition to the gene-specific domains, as described above, the DNA fragments have anchor domains on either side of the gene domain. Anchor domains are domains that are employed in nucleic acid amplification, such as polymerase chain reaction (PCR), steps of the methods, where they serve as primer binding sites for the primers employed in such amplification steps, e.g., as described above. As summarized above, the DNA fragments are also “sample-barcoded”, by which is meant that they include a barcode domain that denotes, i.e., indicates or provides, information about (such that it may be used to determine), the specific sample, e.g., cell, from which the fragment has been produced, where the barcode domains are provided by the barcoded beads of the cell/barcoded bead complexes, e.g., as described above. As reviewed above, barcode domains include unique, specific sequences. While the length of a given barcode domain may vary, in some instances the length ranges from 6 to 60 nt, such as 8 to 40 nt, and including 12 to 20 nt. In addition to the gene-specific, barcode and anchor domains, the fragments produced by methods of the invention may further include additional domains, such as but not limited to a UMI domain, a linker domain, an adaptor domain, etc.

Embodiments of the methods may be characterized as methods of preparing a plurality of sample-barcoded anchor-domain-flanked DNA fragments from a template nucleic acid sample, e.g., a template ribonucleic acid (template RNA) sample. More specifically the methods may be characterized as multiplex methods of preparing a plurality of sample-barcoded anchor-domain-flanked gene-specific deoxyribonucleic acid DNA fragments from a template nucleic acid, e.g., RNA, sample, such that each DNA fragment of the plurality is produced at the same time from the RNA or DNA sample, e.g., each DNA fragment is produced simultaneously from the source RNA or DNA sample. The number of distinct DNA fragments prepared in a given method may vary, where in some instances the number in the plurality ranges from 1 to 200,000, 10 to 25,000, such as 100 to 20,000 and including 1,000 to 10,000, 10,000 to 20,000, 15,000 to 20,000 and 15,000 to 19,000.

Among the DNA fragments of the plurality that are produced from a single sample by methods of the invention, a given DNA fragment is considered to be distinct from another DNA fragment if the gene-specific domains of the two fragments differ from each other by sequence. In some embodiments, the difference between two DNA fragments could be as small as one nucleotide, e.g., gene-specific fragment with single nucleotide polyphormism (SNP) region. While the gene-specific domains of the DNA fragments in a given plurality may all differ from each other, e.g., because they include coding sequences of different genes, the DNA fragments will also include common domains, i.e., domains that are identical to each other (i.e., domains having sequences that do not differ from each other), where these domains are the flanking anchor domains, the barcode domains, etc. When employed, the DNA fragments may further differ with respect to additional domains, such as distinct UMI domains, such that the UMI domains of the DNA fragments have different sequences, i.e., they are not common or identical.

As indicated above, during a given protocol a plurality of DNA fragments produced from one sample may be combined, i.e., pooled, with one or more additional pluralities produced from one or more additional samples, e.g., plurality of single cells or nucleus derived from single cells. In such pooled compositions, each plurality of the pooled composition will have a distinct barcode domain, such that the barcode domain of a first plurality of the composition will have a sequence that differs from every other barcode domain of every other plurality in the pooled composition. In a given pooled composition, each barcode domain has a sequence that is significantly different from that of any other barcode domain in the pooled composition, with a difference of at least 1 nucleotide, such as 2 nucleotides and including 3 or more nucleotide differences in the whole set of barcodes employed in the assay. In this way each plurality of the pooled composition will have a distinct identifying barcode domain. The number of different barcode domains in such pooled compositions is the same as the number of different pluralities in the pooled composition, where the number represents the number of different samples that is employed to make the pooled composition. The number of different barcodes present in a given pooled composition depends on number of samples being analyzed in a given assay. In some instances, the number ranges from 10 to 1,000,000, such as 100 to 100,000, and including 1,000 to 10,000. For example, currently for analysis of single-cell samples, the number of barcodes may be 10,000 or more, but for analysis of clinical samples the number of barcodes may not exceed 1,000.

### Amplification

In some instances, barcoded nucleic acids are amplified, where amplicons are produced from the barcoded nucleic acids produced by the primer extension step, e.g., as described above. The term “amplicon” is employed in its conventional sense to refer to a piece of DNA that is the product of artificial amplification or replication events, e.g., as produced using various methods including polymerase chain reactions (PCR), ligase chain reactions (LCR), rolling circle amplification (RCA), etc. Where primer extension products are amplified, the primer extension products, e.g., as described above, may include additional domains that are employed in subsequent amplification steps to produce a desired amplicon composition. For example, flanking anchor domains are provided in the primer extension products, where the flanking anchor domains include universal priming sites which may be employed in PCR amplification.

As such, embodiments of the methods may include combining a primer extension product composition of barcoded nucleic acids with universal forward and reverse primers under amplification conditions sufficient to produce a desired product barcoded amplicon composition. The forward and reverse universal primers may be configured to bind to the common forward and reverse anchor domains and thereby nucleic acids present in the primer extension product compositions. The universal forward and reverse primers may vary in length, ranging in some instances from 10 to 75 nt, such as 18 to 60 nt.

In some instances, the universal forward and reverse primers include one or more additional domains, such as but not limited to: an indexing domain, a clustering domain, a Next Generation Sequencing (NGS) adaptor domain (i.e., high-throughput sequencing (HTS) adaptor domain), etc. Alternatively, these domains may be introduced during one or more subsequent steps, such as one or more subsequent amplification reactions, e.g., as described in greater detail below. The amplification reaction mixture will include, in addition to the primer extension product composition and universal forward and reverse primers, other reagents, as desired, such polymerase, dNTPs, buffering agents, etc., e.g., as described above.

Amplification conditions may vary. In some instances, the reaction mixture is subjected to polymerase chain reaction (PCR) conditions. PCR conditions include a plurality of reaction cycles, where each reaction cycle includes: (1) a denaturation step, (2) an annealing step, and (3) a polymerization step. The number of reaction cycles will vary depending on the application being performed, and may be 1 or more, including 2 or more, 3 or more, four or more, and in some instances may be 15 or more, such as 20 5 or more and including 30 or more, where the number of different cycles will typically range from about 12 to 24. The denaturation step includes heating the reaction mixture to an elevated temperature and maintaining the mixture at the elevated temperature for a period of time sufficient for any double stranded or hybridized nucleic acid present in the reaction mixture to dissociate. For denaturation, the temperature of the reaction mixture may be raised to, and maintained at, a temperature ranging from 85 to 100° C., such as from 90 to 98° C. and including 94 to 98° C. for a period of time ranging from 3 to 120 sec, such as 5 to 30 sec. Following denaturation, the reaction mixture will be subjected to conditions sufficient for primer annealing to template DNA present in the mixture. The temperature to which the reaction mixture is lowered to achieve these conditions may be chosen to provide optimal efficiency and specificity, and in some instances ranges from about 50 to 75° C., such as 60 to 74° C., and including 68 to 72° C. Annealing conditions may be maintained for a sufficient period of time, e.g., ranging from 10 sec to 30 min, such as from 10 sec to 5 min. Following annealing of primer to template DNA or during annealing of primer to template DNA, the reaction mixture may be subjected to conditions sufficient to provide for polymerization of nucleotides to the primer ends in manner such that the primer is extended in a 5′ to 3′ direction using the DNA to which it is hybridized as a template, i.e. conditions sufficient for enzymatic production of primer extension product. To achieve polymerization conditions, the temperature of the reaction mixture may be raised to or maintained at a temperature ranging from 65 to 75, such as from about 68 to 72° C. and maintained for a period of time ranging from 15 sec to 20 min, such as from 20 sec to 5 min. In some embodiments, the annealing stage could be avoided, and protocol could include only denaturation and polymerization steps as described above. The above cycles of denaturation, annealing and polymerization may be performed using an automated device, typically known as a thermal cycler. Thermal cyclers that may be employed are described in U.S. Pat. Nos. 5,612,473; 5,602,756; 5,538,871; and 5,475,610, the disclosures of which are herein incorporated by reference.

The product amplicon composition of this first amplification reaction will include amplicons corresponding to the gene-specific domains that are present in the initial target nucleic acid composition and are bounded by primer pairs present in the employed set of gene-specific primers and barcode sequence from one side of the amplicon. In some instances, the number of distinct amplicons of differing sequence in this initial amplicon composition ranges from 10 to 19,000, 10 to 15,000, 10 to 10,000, and 10 to 8,000, such as 25 to 18,500, 25 to 12,000, 25 to 8,000, and 25 to 7,500, including 50 to 15,000, 50 to 10,000 and 50 to 5,000, where in some instances the number of distinct amplicons present in this initial amplicon composition is 25 or more, including 50 or more, such as 100 or more, 250 or more, 500 or more, 1,000 or more, 1,500 or more, 2,500 or more, 5,000 or more, 7,500 or more, 8,500 or more, 10,000 or more, 15,000 or more, 18,000 or more. A subject amplicon composition may include or exclude multiple different product amplicons corresponding to same gene as amplified by two or more different primer pairs directed to the gene. The multiple product amplicons making up the amplicon composition may vary in length, ranging in length in some instances from 50 to 1000, such as 60 to 500, including 70 to 250 nt.

The sample barcoded initial product amplicon composition may be employed in a variety of different applications, including mutation, copy number, somatic variation in genomic DNA. In other embodiments, the barcoded amplicon composition can be used for evaluation of the expression profile of the sample from which the template target nucleic acid was obtained. In such instances, the expression profile may be obtained from the amplicon composition using any convenient protocol, such as but not limited to differential gene expression analysis, array-based gene expression analysis, NGS sequencing, etc.

For example, the barcoded amplicon composition may be employed in hybridization assays in which a nucleic acid array that displays “probe” nucleic acids for each of the genes to be assayed/profiled in the profile to be generated is employed. In these assays, the amplicon composition is first prepared from the initial target nucleic acid sample being assayed as described above, where preparation may include labeling of the target nucleic acids with a label, e.g., a member of signal producing system.

Following amplicon production, e.g., as described above, the sample is contacted with the array under hybridization conditions, whereby complexes are formed between target nucleic acids that are complementary to probe sequences attached to the array surface. The presence of hybridized complexes is then detected, either qualitatively or quantitatively. The detection and quantification of different barcodes could be achieved in the follow-up hybridization steps with labeled targets complementary to barcode domains of the amplicons. Specific hybridization technology which may be practiced to generate the expression profiles employed in the subject methods includes the technology described in U.S. Pat. Nos. 5,143,854; 5,288,644; 5,324,633; 5,432,049; 5,470,710; 5,492,806; 5,503,980; 5,510,270; 5,525,464; 5,547,839; 5,580,732; 5,661,028; 5,800,992; the disclosures of which are herein incorporated by reference; as well as WO 95/21265; WO 96/31622; WO 97/10365; WO 97/27317; EP 373 203; and EP 785 280. In these methods, an array of “probe” nucleic acids that includes a probe for each of the phenotype determinative genes whose expression is being assayed is contacted with target nucleic acids as described above. Contact is carried out under hybridization conditions, e.g., stringent hybridization conditions, and unbound nucleic acid is then removed. The resultant pattern of hybridized nucleic acid provides information regarding expression for each of the genes that have been probed, where the expression information is in terms of whether or not the gene is expressed and, typically, at what level, where the expression data, i.e., expression profile (e.g., in the form of a transcriptome), may be both qualitative and quantitative.

Alternatively, non-array-based methods for quantifying the levels of one or more nucleic acids in a sample may be employed, including quantitative PCR, real-time quantitative PCR, and the like. (For general details concerning real-time PCR see Real-Time PCR: An Essential Guide, K. Edwards et al, eds., Horizon Bioscience, Norwich, U.K. (2004)).

In some embodiments, the method further includes sequencing the multiple barcoded product amplicons, e.g., by using a Next Generation Sequencing (NGS) protocol. In such instances, if not already present, the methods may include modifying the initial amplicon composition to include one or more components employed in a given NGS protocol, e.g., sequencing platform adaptor constructs, indexing domains, clustering domains, etc.

By “sequencing platform adapter construct” is meant a nucleic acid construct that includes at least a portion of a nucleic acid domain (e.g., a sequencing platform adapter nucleic acid sequence) or complement thereof utilized by a sequencing platform of interest, such as a sequencing platform provided by Illumina® (e.g., the NovaSeq™, NexSeq™, HiSeq™, MiSeg™ and/or Genome Analyzer™ sequencing systems); Thermo Fisher (e.g., Ion Torrent™ (such as the Ion PGM™ and/or Ion Proton™ sequencing systems) and Life Technologies™ (such as a SOLiD sequencing system)); Pacific Biosciences (e.g., the PACBIO RS II sequencing system); Roche (e.g., the 454 GS FLX+ and/or GS Junior sequencing systems); Oxford Nanopore technologies (e.g., MinION™, GridION™, PrometION™ sequencing systems) or any other sequencing platform of interest.

In certain aspects, the sequencing platform adapter construct includes a nucleic acid domain selected from: a domain (e.g., a “capture site” or “capture sequence”) that specifically binds to a surface-attached sequencing platform oligonucleotide (e.g., the P5/i5 or P7/i7 oligonucleotides attached to the surface of a flow cell in an Illumina® sequencing system); where the construct may include one or more additional domains, such as but not limited to: a sequencing primer binding domain or clustering domain (e.g., a domain to which the Read 1 or Read 2 primers of the IIlumina® platform may bind); a indexing domain (e.g., a domain that uniquely identifies the sample source of the nucleic acid being sequenced to enable sample multiplexing by marking every molecule from a given sample with a specific index or “tag”); a barcode sequencing primer binding domain (a domain to which a primer used for sequencing a barcode binds); a unique molecular identification domain (e.g., a molecular index tag, such as a randomized tag of 4, 6, or other number of nucleotides) for uniquely marking molecules of interest to determine expression levels based on the number of instances a unique tag is sequenced; a complement of any such domains; or any combination thereof. In certain aspects, a barcode domain (e.g., sample index tag) and a molecular identification domain (e.g., a molecular index tag) may be included in the same nucleic acid.

The sequencing platform adapter constructs may include nucleic acid domains (e.g, “sequencing adapters”) of any length and sequence suitable for the sequencing platform of interest. In certain aspects, the nucleic acid domains are from 4 to 200 nucleotides in length. For example, the nucleic acid domains may be from 4 to 100 nucleotides in length, such as from 6 to 75, from 8 to 50, or from 10 to 40 nucleotides in length. According to certain embodiments, the sequencing platform adapter construct includes a nucleic acid domain that is from 2 to 8 nucleotides in length, such as from 9 to 15, from 16-22, from 23-29, or from 30-36 nucleotides in length. The nucleic acid domains may have a length and sequence that enables a polynucleotide (e.g., an oligonucleotide) employed by the sequencing platform of interest to specifically bind to the nucleic acid domain, e.g., for solid phase amplification and/or sequencing by synthesis of the cDNA insert flanked by the nucleic acid domains. Example nucleic acid domains include the P5 (5′-AATGATACGGCGACCACCGA-3′) (SEQ ID NO:03), P7 (5′-CAAGCAGAAGACGGCATACGAGAT-3′) (SEQ ID NO:04), Read 1 primer (5′-ACACTCTTTCCCTACACGACGCTCTTCCGATCT-3′) (SEQ ID NO:05) and Read 2 primer (5′-GTGACTGGAGTTCAGACGTGTGCTCTTCCGATCT-3′) (SEQ ID NO:06) domains employed on the Illumina®-based sequencing platforms. Other example nucleic acid domains include the A adapter (5′-CCATCTCATCCCTGCGTGTCTCCGACTCAG-3′) (SEQ ID NO:07) and P1 adapter (5′-CCTCTCTATGGGCAGTCGGTGAT-3′) (SEQ ID NO:08) domains employed on the Ion Torrent™-based sequencing platforms.

The nucleotide sequences of nucleic acid domains useful for sequencing on a sequencing platform of interest may vary and/or change over time. Adapter sequences are typically provided by the manufacturer of the sequencing platform (e.g., in technical documents provided with the sequencing system and/or available on the manufacturer's website). Based on such information, the sequence of the sequencing platform adapter construct of the template switch oligonucleotide (and optionally, a first strand synthesis primer, amplification primers, and/or the like) may be designed to include all or a portion of one or more nucleic acid domains in a configuration that enables sequencing the nucleic acid insert (corresponding to the template nucleic acid) on the platform of interest.

The sequencing adaptors may be added to the amplicons of the initial amplicon composition using any convenient protocol, where suitable protocols that may be employed include, but are not limited to: amplification protocols, ligation protocols, etc. In some instances, amplification protocols are employed. In such instances, the initial amplicon composition may be combined with forward and reverse sequencing adaptor primers that include one or more sequencing adaptor domains, e.g., as described above, as well as domains that bind to universal primer sites found in all of the amplicons in the composition, e.g., the forward and reverse anchor domains, such as described above. As reviewed above, amplification conditions may include the addition of forward and reverse sequencing adaptor primers configured to bind to the common forward and reverse anchor domains and thereby amplify all or a desired portion of the product nucleic acid, dNTPs, and a polymerase suitable for effecting the amplification (e.g., a thermostable polymerase for polymerase chain reaction), where examples of such conditions are further described above. The forward and reverse sequencing adaptor primers employed in these embodiments may vary in length, ranging in length in some instances from 20 to 60 nt, such as 25 to 50 nt. Addition of NGS sequencing adaptors results in the production of a composition which is configured for sequencing by an NGS sequencing protocol, i.e., an NGS library.

In certain aspects, the methods of the present disclosure further include subjecting the NGS library to NGS protocol, e.g., as described above. The NGS protocol will vary depending on the particular NGS sequencing system employed. Detailed protocols for sequencing an NGS library, e.g., which may include further amplification (e.g., solid-phase amplification), sequencing the amplicons, and analyzing the sequencing data are available from the manufacturer of the NGS system employed. Protocols for performing next generation sequencing, including methods of processing the sequencing data, e.g., to count and tally sequences and assemble transcriptome data therefrom, are further described in published United States Patent Application 20150344938, the disclosure of which is herein incorporated by reference.

### Pooling

Where desired, a given workflow may include a pooling step where a product composition, e.g., made up of hybridized barcoded gene-specific primer-RNA complexes, synthesized first strand cDNAs or synthesized double stranded cDNAs, is combined or pooled with product compositions obtained from one or more additional samples, e.g., cells. In some instances, for single-cell analysis the pooling step is performed just after hybridization step between barcoded gene-specific primers and target nucleic acids, e.g., as reviewed above. The number of different product compositions produced from different samples, e.g., cells, that are combined or pooled in such embodiments may vary, where the number ranges in some instances from 2 to 1,000,000, such as 3 to 200,000, including 4 to 100,000 such as 5 to 50,000, where in some instances the number ranges from 100 to 10,000, such as 1,000 to 5,000. Prior to or after pooling, the product composition(s) can be amplified, e.g., by polymerase chain reaction (PCR), such as described above.

### Gene-Specific Primer Protocols

As reviewed above, in some instances gene-specific reverse and forward primers may be employed. Aspects of such embodiments include employing a set of gene-specific primer pairs, wherein each pair of gene-specific primers is made up of a forward primer and a reverse primer, at least one of which includes a sample barcode domain. Examples of sets of reverse gene-specific primers and their use in single cell genetic analysis applications are disclosed in U.S. patent application Ser. Nos. 15/133,184 and 16/543,211, the disclosures of which sets of gene-specific reverse primers are incorporated herein by reference.

### Utility

The subject methods find use in a variety of applications, including expression profiling or transcriptome determination applications, where a sample is evaluated to obtain an expression profile of the sample. By “expression profile” is meant the expression level of a gene of interest in a sample, which may be a single cell or a combination of multiple cells (e.g., as determined by quantitating the level of an RNA or protein encoded by the gene of interest), or a set of expression levels of a plurality (e.g., is 2 or more) of genes of interest. In certain aspects, the expression profile includes expression level data for 1, 2 or more, 5 or more, 10 or more, 20 or more, 50 or more, 100 or more, 200 or more, 300 or more, 400 or more, 500 or more, 1,000 or more, 5,000 or more, 10,000 or more, 15,000 or more, e.g., 18,000 or more genes of interest. According to one embodiment, the expression profile includes expression level data of from 50 to 8000 genes of interest, e.g., from 1000 to 5000 genes of interest. In some embodiments, the expression profile includes expression level data of from 50 to 19,000 genes of interest, e.g., from 1000 to 18,000 genes of interest. In certain aspects, the methods may be employed detecting and/or quantitating the expression of all or substantially all of the genes transcribed by an organism, e.g., a mammal, such as a human or mouse, in a target cell. The terms “expression” and “gene expression” include transcription and/or translation of nucleic acid material. For example, gene expression profiling may include detecting and/or quantitating one or more of any RNA species transcribed from the genomic DNA of the target cell, including pre-mRNAs, mRNAs, non-coding RNAs, microRNAs, small RNAs, regulatory RNAs, and any combination thereof.

Expression levels of an expressed sequence are optionally normalized by reference or comparison to the expression level(s) of one or more control expressed genes, including but not limited to, ACTB, GAPDH, HPRT-1, RPL25, RPS30, and combinations thereof. These “normalization genes” have expression levels that are relatively constant among target cells in the cellular sample.

In some instances, quantitative analysis of gene expression using set of calibration control template composition is performed. Internal calibration control templates which mimic but differ from natural target RNAs and spiked into cell or cell lysates at specific amount may be effectively used for truly quantitative expression analysis. The calibration control RNAs could be developed for the set of genes (e.g. cell marker genes) or for genome-wide set of transcripts. In order to address the reproducibility of the profiling assay for multiple biological samples (e.g. thousands of single cells), embodiments of the invention uniquely employ the strategy of using barcoded reverse gene-specific primers. Target template RNAs (e.g., present in cell is extracts) hybridized with barcoded reverse gene-specific primers could be combined for the all follow-up steps. The strategy of barcoding and combining target RNAs at early (hybridization) stage allows for significantly reduced cost of the assay, eliminates sample-to-sample profiling variability due to differences in experimental assay conditions, etc. The developed protocol which addresses sample-to-sample and batch effect variability has significant utility in biomarker discovery in clinical samples (e.g., whole blood).

According to certain embodiments, the expression profile includes “binary” or “qualitative” information regarding the expression of each gene of interest in a target cell. That is, in such embodiments, for each gene of interest, the expression profile only includes information that the gene is expressed or not expressed (e.g., above an established threshold level) in the sample being analyzed, e.g., tissue, cell, etc. In other embodiments, the expression profile includes quantitative information regarding the level of expression (e.g., based on rate of transcription, rate of splicing and/or RNA abundance) of one or more genes of interest. A qualitative and/or quantitative expression profile from the sample may be compared to, e.g., a comparable expression profile generated from other samples and/or one or more reference profiles from cells known to have a particular genotype, biological phenotype or condition (e.g., cellular DNA with a specific natural or engineered mutation, a disease condition, such as a tumor cell; or treatment condition, such as a cell treated with an agent, e.g., a drug). When the profiles being compared are quantitative expression profiles, the comparison may include determining a fold-difference between one or more genes in the expression profile of a target cell and the corresponding genes in the expression profile(s) of one or more different target cells in the cellular sample, or the corresponding genes in a reference cell or cellular sample. Alternatively, or additionally, the expression profile may include information regarding the relative expression levels of different genes in a single target cell. In certain aspects, the fold difference in intercellular expression levels or intracellular expression levels can be determined to be 0.1 or more, 0.5 fold or more, 1 fold or more, 1.5 fold or more, 2 fold or more, 2.5 fold or more, 3 fold or more, 4 fold or more, 5 fold or more, 6 fold or more, 7 fold or more, 8 fold or more, 9 fold or more, or more than 10 fold or more, for example.

In some instances, the methods may be employed to determine the transcriptome of a sample. The term “transcriptome” is employed in its conventional sense to refer to the set of all messenger RNA molecules in one cell or a population of cells. In some instances, a transcriptome includes the amount or concentration of each RNA molecule in addition to the molecular identities. The methods described herein may be employed in detecting and/or quantitating the expression of all genes or substantially all genes of the transcriptome of an organism, e.g., a mammalian organism, such as a human or a mouse, for a particular target cell or a population of cells.

Expression profiles obtained using methods of the invention may be employed in a variety of applications. For example, an expression profile may be indicative of the biological condition of the sample or host from which the sample is obtained, including but not limited to a disease condition (e.g., a cancerous condition, metastatic potential, an epithelial mesenchymal transition (EMT) characteristic, and/or any other disease condition of interest), the condition of the cell in response to treatment with any physical action (e.g., heat shock, hypoxia, normoxia, hydrodynamic stress, radiation, and/or the like), the condition of the cell in response to treatment with chemical compounds (e.g., drugs, cytotoxic agents, nutrients, salts, and/or the like) or biological extracts or entities (e.g., viruses, bacteria, other cell types, growth factors, biologics, and/or the like), and/or any other biological condition of interest (e.g. immune response, senescence, inflammation, motility, and/or the like).

Embodiments of the invention find further application in tumor microenvironment analysis applications. Transcriptome data obtained, e.g., as described above, may be employed to determine the cellular composition of a tumor sample, e.g., to provide an evaluation of the types of cells present in a tumor sample, such as infiltrating hematopoietic cells, tumor cells and bulk tissue cells. For example, transcriptome data may be employed to assess whether a tumor sample does not include infiltrating immune cells, including those of the adaptive and/or innate immune system, such as but not limited to: T, B, natural killer, monocyte, granulocytes, neutrophils, basophils, platelets, and their myeloid and lymphoid progenitor cells, hematopoietic stem cells, and the like. Such information may be used, e.g., in therapy determination applications, for example where the presence of infiltrating immune cells indicates that a patient will be responsive to immunotherapy while the absence of infiltrating immune cells indicates that a patient will not be responsive to immunotherapy. As such, aspects of the invention include methods of therapy determination, where a patient tumor sample is evaluated to assess the tumor microenvironment. Aspects of the invention may further include making a determination to employ an immunotherapy protocol is made if the tumor microenvironment includes infiltrating tumor cells and a determination is made to employ a non-immunotherapy treatment regimen if the tumor microenvironment lacks infiltrating immune cells.

Methods as described here also find use in large-scale profiling of single-cell phenotypes derived from model system (e.g., cultivated cells, organoid cultures, 3D cultures, etc.), model organisms (e.g., mice, rat, monkey, etc.) and clinical samples derived from normal or pathological conditions (e.g., blood, biopsy, sputum, saliva, etc,). Currently, there is a substantial need for comprehensive characterization of different cell types present in normal and pathological conditions. The disclosed methods and compositions provide an improved technological platform for large-scale discovery of key cellular markers for developing novel diagnostic and prognostic tools.

Transcriptome data, e.g., produced as described above, also finds use in other non-clinical applications, such as predictive and prognostic biomarker discovery applications, evaluation of cancer immunoediting mechanism applications, drug target discovery, and the like.

In other embodiments, the gene expression level measurement can be combined with profiling of genotype or genetic changes in the target cells. Genetic changes of interest include both natural changes, e.g., those present in cells derived from biological sources, and engineered modifications in target cells, e.g., in genomic DNA. Examples of natural mutation are single nucleotide polymorphism (SNP), copy number variation (CNV), deletions, translocation, gene fusions, recombinations, etc., which may be associated with development of disease state (e.g., cancer, genetic diseases, etc.) in normal cells. Engineered genetic changes may be generated by a wide range of genetic engineered methods (e.g., delivery of constructs by viral, plasmid vectors, synthetic DNA and RNA constructs, etc.) and include, but are not limited to, gene editing (base editing, homologous recombination, etc.), delivery and expression of effector constructs (sgRNA, shRNA, peptides, proteins, aptamers, microRNA, asRNA, etc.) and the like. Usually, effector constructs could change expression (e.g., activation, repression, inactivation, etc.) of target genes. Other types of genetic constructs which do not change expression of genes but may be employed for cell tracking (clonal barcodes or UMI), measure expression of proteins (e.g., antibody-barcoded oligonucleotide constructs), signaling pathway (transcriptional reporter vectors), and other biological processes (e.g., regulation of immune functions, apoptosis, etc.) may also be employed. In some applications the genetic changes may be identified by the disclosed invention in episomal DNA or in genomic DNA, e.g., if a genetic construct is integrated in genomic DNA. In other applications the genetic changes or effector constructs may be transcribed and profiled by designing gene-specific primers specific for both effector and transcribed cellular RNAs. Importantly, the disclosed methods of multiplex PCR may generate both expression profile and identify genetic changes or/and effectors in a single assay and therefore characterize and link the phenotype of the cells with specific genetic changes. For example, the combination of expression profiles with identification of effectors (sgRNA, shRNA, etc.) which could induce knock-out, knock-down or activation of target genes allows one to characterize the functions of these genes hi normal and disease state. Simultaneous profiling of the natural or induced mutations and transcriptome allows one to find and characterize mechanisms of driver mutations critical for development of disease states (e.g., cancer, senescence, etc.). Monitoring cell phenotypes by expression profiling of different cell clones (e.g., with different mutations labeled by different barcodes) under different growth conditions (cellular environment, drug treatment) allows one to identify rare cancer stem or drug resistant cells.

### Compositions

Aspects of the invention further include various compositions. Compositions of the invention may include, e.g., one or more of any of the reaction mixture components described above with respect to the subject methods. For example, the compositions necessary for generation of cell/barcoded bead complexes may include individual cells or group of cells, barcoded beads, including barcoded beads with a cell binding moiety, buffers necessary for binding and purification of cell/barcoded bead complexes, and the like. In some embodiments, additional components comprising consumables and reagents may be included in composition. The composition necessary for generation of primed template nucleic acids may include components like cell media (e.g., PBS), hybridization buffer (e.g., 1× TCL, etc.) and lysis buffer (e.g., 0.2% NP-40) as described in a details above. Additional components which could be used to increase efficiency, specificity and rate of cell lysis, hybridization (e.g., salts or polynucleotides) and barcoded primer releasing reagents (e.g., DTT) may also be included in the composition. The components necessary for pooling and purification of primed template nucleic acids including oligo dT magnetic beads, AMPure beads, Silica beads, washing and elution buffers, etc. may be included in compositions. Compositions necessary for generation of barcoded nucleic acids and barcoded amplicon compositions may include a primed template nucleic acid, polymerase (e.g., a reverse transcriptase and thermostable DNA polymerase), dsDNAse, single-stranded nuclease (e.g. exonuclease I) a set of gene-specific, anchor PCR and indexed NGS primers, dNTPs, a polymerase, buffers, a metal cofactor, one or more nuclease inhibitors (e.g., an RNase inhibitor), one or more enzyme-stabilizing components (e.g., DTT), or any other desired reaction mixture component(s). Composition may vary for the different steps of the disclosed methods. For example, for cDNA synthesis steps the compositions may include only reagents necessary for reverse transcription (e.g., reverse transcriptase) and for the subsequent primer extension and amplification step the composition may employ a different buffer, oligonucleotides and enzymes (DNA polymerase) components. Also provided are compositions that include a barcoded primer extension product composition, e.g., as described above. Also provided are barcoded amplicon compositions and NGS libraries, such as described above.

The subject compositions may be present in any suitable environment. According to one embodiment, the compositions are present in reaction tubes (e.g., a 0.2 mL tube, a 0.5 mL tube, a 1.5 mL tube, or the like), a well (e.g. 6-, 24-, or 96-well plates), and a vials (e.g., 5, 10, 50, 200 mL bottles). In certain aspects, the compositions are present in two or more (e.g., a plurality of) reaction tubes or wells (e.g., a plate, such as a 96-well plate). The tubes and/or plates and/or vials may be made of any suitable material, e.g., polypropylene, or the like. In certain aspects, the tubes and/or plates in which the composition is present provide for efficient heat transfer to the composition (e.g., when placed in a heat block, water bath, thermocycler, and/or the like), so that the temperature of the composition may be altered within a short period of time, e.g., as necessary for a particular cell lysis, hybridization, or enzymatic reaction to occur. According to certain embodiments, the composition is present in a thin-walled polypropylene tube, or a plate having thin-walled polypropylene wells. Other suitable environments for the subject compositions include, e.g., a microfluidic chip (e.g., a “lab-on-a-chip device”). The composition may be present in an instrument(s) configured to analyze composition of cell/barcoded bead complexes (e.g., microscope with image analysis functions), treat the composition with physical stimulus (e.g., UV light) or bring the composition to a desired temperature, e.g., a temperature-controlled water bath, heat block, or the like. The instrument configured to bring the composition to a desired temperature may be configured to bring the composition to a series of different desired temperatures, each for a suitable period of time (e.g., the instrument may be a thermocycler).

### Kits

Aspects of the present disclosure also include kits. The kits may include, e.g., one or more of any of the reaction mixture components described above with respect to the subject methods. For example, the kits may include one or more of: a set of barcoded reverse gene-specific primers immobilized on beads, a polymerase (e.g., a thermostable polymerase, a reverse transcriptase both with hot-start properties, or the like), dsDNAse, exonuclease, dNTPs, a metal cofactor, one or more nuclease inhibitors (e.g., an RNase inhibitor and/or a RNase inhibitor), one or more molecular crowding agents (e.g., polyethylene glycol, or the like), one or more enzyme-stabilizing components (e.g., DTT), a stimulus response polymer, or any other desired kit component(s), such as solid supports, containers, cartridges, e.g., tubes, beads, plates, microfluidic chips, etc.

Components of the kits may be present in separate containers, or multiple components may be present in a single container. For example, the individual barcoded oligonucleotides could be provided pre-aliquoted in separate wells or attached/encapsulated with different beads, and mixture of all barcoded beads is provided as kit components. In certain embodiments, it may be convenient to provide the components in a lyophilized form, so that they are ready to use and can be stored conveniently at room temperature.

In addition to the above-mentioned components, a subject kit may further include instructions for using the components of the kit, e.g., to practice the subject method. The instructions are generally recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or subpackaging) etc. In other embodiments, the instructions are present as an electronic storage data file present on a suitable computer readable storage medium, e.g. CD-ROM, diskette, Hard Disk Drive (HDD), portable flash drive, etc. In yet other embodiments, the actual instructions are not present in the kit, but means for obtaining the instructions from a remote source, e.g. via the internee, are provided. An example of this embodiment is a kit that includes a web address where the instructions can be viewed and/or from which the instructions can be downloaded. As with the instructions, this means for obtaining the instructions is recorded on a suitable substrate.

The following examples are offered by way of illustration and not by way of limitation.

### EXPERIMENTAL

### Example 1. Protocol for Producing Sets of Experimentally Validated Gene-Specific Primers

### A. Primer Design.

Briefly, our primer design pipeline consists of four major steps: (1) identify all primer binding-site positions among all possible DNA/RNA target template sequences; (2) evaluate the binding stability of the entire primer sequence using the thermodynamic model to calculate the duplex stability; (3) filter amplicons by size and target region position and (4) in silico designed primer pairs are experimentally validated using primers/corresponding target regions and used under a common PCR thermal profile, facilitating the evaluation of target transcripts of a large number genes in parallel using Next Generation Sequencing (NGS).

General rules concerning optimal primer length, CG content, annealing and melting temperature, secondary structure issues are included. Since oligonucleotide primers need to be specific and provide the optimal annealing and melting temperatures, primers of 20-30 nt are considered to be the best for forward gene specific primer target sequence extension reactions in target regions and GC content of >50%<75%. Importantly, reverse gene specific primers designed for RNA/DNA hybridization step and follow-up extension step are usually longer (e.g. 25-40 nt) in order to provide higher stability of hybridization complex between target template and primer.

### B. Design and Synthesis of Barcoded Reverse and Forward Gene Specific Primers.

Barcoded reverse gene specific primers are assembled by ligation of a pool of reverse gene specific primers with barcoded oligonucleotides immobilized on the surface of beads:

```
(from right to left SEQ ID NOs: 09-11)
3′ L2 5′ 3′ L1
  RevGSP-ACCGACCAGCACCp GCCAGCACGCCA-(Barcode)-
          Anchor 2 5′
AGACACGACCAGCCACGAGCA-X-Bead
(SEQ ID NO: 12)
A-TGGCTGGTCGTGG--CGGTCGTGCGGT-3′dT
Link1s
```

Barcoded oligonucleotides with minimum structure: linker 5′-Anchor 2-Barcode-Linker L1-3′ are ligated to reverse gene specific primer set (RevGSP) with minimum structure 5′-phosphate-Linker L2-RevGSP-3′ using complementary to linker Li and linker L2 oligonucleotide Link1s and DNA ligase under ligation conditions. DNA ligation reaction attaches barcoded anchor oligonucleotides to reverse gene specific primers. In another protocol, the ligation is used to attach both barcodes and CSP to the beads (e.g. polyacrylamide) comprising anchor oligonucleotides. The bead-anchor conjugates may be generated by free radical polymerization reaction in a microfluidics device of acrylamide/bis-acrylamide and anchor oligonucleotide with acrydite modification at the 5′-end. As a result of polymerization reaction anchor oligonucleotide is incorporated in polyacrylamide matrix and could be used for follow-up ligation steps with linker-barcoded oligonucleotides. Specifically, the anchor-bead conjugates are split into multiple wells (e.g., 1,000 or more) wherein each well has uniquely barcoded oligonucleotide which is ligated to anchor with the help of complementary to linker and anchor oligonucleotide. After first ligation step, the beads with barcoded anchors are washed, mixed together, split again for multiple wells (e.g., 1,000 or more) and ligated again with the second set of uniquely barcoded oligonucleotides using oligonucleotide complementary to common linker sequence. After the second ligation step, the barcoded beads are washed, mixed together and finally ligated for RevGSP as described above. The disclosed protocol may be used for beads which cannot be used in oligonucleotide chemical synthesis, like hydrogel bead compositions. In an alternative strategy, antisense RevGSP-L1 complement oligonucleotides are synthesized, annealed to L1-barcoded oligonucleotides and extended by Klenov DNA polymerase. As a result of ligation (or primer extension) reaction, the set of reverse gene specific primers will be labeled with specific barcode. The set of barcoded reverse gene specific primers is purified from non-ligated products by washing the bead-barcoded RevGSP conjugates in 0.1 M of NaOH and used in disclosed primer extension assay. The same set of gene specific primers could be labeled with plurality of different barcodes using the same protocol. In another embodiment, the same protocol could be used for barcoding set of forward gene specific primers.

Barcode-Anchor oligonucleotides maybe attached to the solid surface (e.g. beads) through linker X (e.g. X could be a cleavable linker). Furthermore, the different binding moiety (e.g. antibodies) may be attached to the beads to provide binding of Antibody-Bead-barcoded GSP complex to specific cell type through antigen-antibody interaction. Importantly, each barcode could have a complex structure as described in the application in more detail. These complex composite barcodes could have several domains, including but not limited to:
1) Sample barcode—specific sequence (usually from 8-14 nt) attached to a set of gene-specific primers which allow for labeling of all extension products derived from target RNA sample.
2) Universal molecular identifier (UMI)—complex random, semi-random (usually 8-12 nt), or set of unique specific sequences which allow for labeling of each molecule used in disclosed primer extension assay with unique sequence/barcode. UMI could be added to RevGSP-Linker 2 set between RevGSP and linker L2.
3) Bead barcode—specific sequence (10-16 nt) unique for each bead if gene-specific primers are attached to the beads. In some embodiments, e.g. for single cell analysis applications (e.g. if only one biological sample used in the assay), bead barcode could be sample barcode.
4) Antibody barcode—specific sequence unique for each specific antibody immobilized to the beads.
   Linker L1, linker L2 and complementary Link1s could be designed with variety of different sequences with minimum length of L1 and L2 are 4 nt each. Linkers which separate different barcodes generated by DNA-ligation step may be at least 4 nt, as desired.
   Examples of Anchor2-Barcode-Linker 1 oligonucleotides used in ligation reaction:
   Barcodes are underlined

Ana-BC1-L1
(SEQ ID NO: 13)
ACGAGCACCGACCAGCACAGAGAACAAACACCGCACGACCG
Ana-BC2-L1
(SEQ ID NO: 14)
ACGAGCACCGACCAGCACAGAGGCGAAACACCGCACGACCG
Anc2-BC3-L1
(SEQ ID NO: 15)
ACGAGCACCGACCAGCACAGAGCAAAAGGACCGCACGACCG

Example of Bead-barcoded oligonucleotide conjugates (synthesized by Chemgenes, Inc.) used in ligation reaction.

In the diagram below: PClinker—photocleavable linker, or SSlinker—bi-sulfite linker cleaved by sulfite ions (e.g. DTT treatment) is used for detachment of reverse barcoded gene specific primers from the beads; Anchor2—binding site for universal amplification primer; UMI—universal molecular index; Barcode—sample-specific 6 nt barcode (underlined); Linker L2-sequence necessary for ligation of barcodes with gene specific primer set; bead—polystyrene or hydrogel beads with sizes 10-100 microns.

```
ChemB-PC1-Anc2-BC-L2
(SEQ ID NO: 16)
Bead-linker-PClinker-
                        Anchor 2 UMI Barcode Linker L2
AGCACCGACCAGCACAGAVVNVVNVVCATCAGACCGCACGACCG-3′
ChemB-SS-Anc2-BC-L2
(SEQ ID NO: 17)
Bead-linker-SSlinker-
                        Anchor 2 UMI Barcode Linker L2
AGCACCGACCAGCACAGAVVNVVNVVCAGCATGACCGCACGACCG-3′
```

Example of final barcoded reverse gene specific primer structure employed in the assay

Anchor2 L1-L2 linker
(from left to right SEQ ID NOs: 09 and 18)
5′-ACGAGCACCGACCAGCACAGA-(UMI-Barcode)-ACCGCACGACCGCCACGACCAGCCA-RevGSP-3′

Wherein, L2-L1 linker sequence generated by ligation of L1 and L2 linkers, Barcode—complex barcode, and UMI—universal molecular index as described in a more detail above, Anchor2—universal primer binding site.

In some embodiments, the barcoded reverse gene specific primer composition could be synthesized by combinatorial (pool and split) chemical synthesis without DNA ligation step. In this embodiment, L2-L1 linker will be missing in the final structure.

Similar structure could be generated for barcoded forward gene specific primer set and employed in the disclosed assay:

Anchor1 L1-L2 linker
(from left to right SEQ ID NOs: 19 and 18)
5′-AGCACCGACCAGCAGACA-(UMI-Barcode)-ACCGCACGACCGCCACGACCAGCCA-RevGSP-3′

In other embodiments, the forward gene specific primers are designed and used in the assay without barcoded and synthesized by conventional oligonucleotide synthesis with the following structure:

Anchor1
(SEQ ID NO: 20)
5′-AGCACCGACCAGCAGACA-FwdGSP-3′

### C. High-Throughput Gene Specific Primer Validation

Multiplex PCR primers with target template binding domain sequences are screened en masse. In some embodiments, the set of barcoded reverse gene specific primers (with the structure shown above) is first hybridized to control natural or synthetic template RNAs. Furthermore, the hybrids between target mRNA and barcoded reverse gene specific hybrids are combined together, purified and used as mix in the follow-up primer extension and amplification steps. In some embodiments, the hybridization step is performed with RNA sample and barcoded reverse gene specific primers in solution (e.g. primers released from beads). As discussed in a more detail above, the selection of primers with high hybridization efficiency, specificity to specifically hybridize to target sequences and stability of target mRNA-primer complexes is the critical step which defines the overall performance of the assay and cross-talk between different samples. Moreover, using the barcoded reverse primers in the first step of protocol allows one to combine all samples together and therefore scale-up the assay for analysis of hundreds-thousands of samples in the single test tube format.

Uniformity of amplification, including primer efficiency, primer specificity and dynamic range (minimum 100-fold) are determined from multiplex reaction kinetic data. In order to reliably measure expression of different genes, the panel of 15 different human universal RNA from different commercial sources (Agilent, Clontech, BioChain, Qiagen, etc.) and synthetic template RNA are used as templates for cDNA synthesis. Non-specific primer activities are measured by yield of non-targeted products from human universal RNAs and negative control templates (human genomic DNA and mouse universal RNAs). The protocol for testing primer performance is repeated several times with a set of 3-5 PCR primer pairs per gene until the primers with high specific and low non-specific activity are selected. Finally, functionally validated primers are selected as experimentally validated primers for use in sets of experimental validated gene specific primers.

### Example 2. Development of Barcoded Beads with Cell Binding Moiety

To develop barcoded beads with cell binding moiety several strategies may be employed. In the first approach, the chimeric oligonucleotide with structure:

L2
(SEQ ID NO: 21)
5′-pCCACGACCAGCCA-Moiety

is synthesized by conventional phosphoramidite chemistry and ligated to barcoded beads using the protocol described in a more detail above. Examples of cell binding moieties which may be synthesized as oligonucleotide conjugates include lipids (cholesterol, fatty acid, like stearoylic or palmitic acid), oligonucleotide aptamers, e.g. CD4 aptamer with structure:

(SEQ ID NO: 22)
5′-CCACCACCGTACAATTCGCTTTCTTTTTTCATTACCTACTCTGGC-3′

In the second approach, the non-specific (e.g. against beta2-microglobulin, CD293) or cell-type specific (e.g. against CD4, CD8, CD19, etc.) antibodies are conjugated to barcoded beads through covalent or non-covalent bonds. In one protocol the antibodies with cell binding properties are bound to beads (e.g. polystyrene beads) though passive adsorption. In another protocol, antibodies are bound to beads using amino-modified linker domain (with structure: 5′-pCCACGACCAGCCA-NH2-3′) (SEQ ID NO:23) ligated to barcoded beads) and click chemistry. In this protocol the antibodies and amino-modified barcoded beads are activated and conjugated using click chemistry (ThunderLink kit, Expedion) using manufacturer's protocol. In another protocol, the amino-modified oligonucleotide complementary to L21-L2 linker domain:

(SEQ ID NO 24)
5′NH2-TGGCTGGTCGTGGCGGTCGTGCGGT-3′

is conjugated with antibodies using conventional click chemistry regents and manufacturer's protocol (ThunderLink kit, Expedion). The antibody-Linker complement conjugates are incubated with barcoded beads in buffer comprising 50 mM TrisHCl, ph 7.8, 1 M NaCl, 0.1% Tween20 at 120 for 3 hours and purified from non-bind antibodies by using washing in 1× PBS solution and centrifugation steps.

### Example 3. Binding of Barcoded Beads with Cell Sample and Enrichment for Single Cell-Single Bead Complexes in Solution

The barcoded beads with cell binding moiety are washed in 1× PBS and bound with single cells at ratio 1.5-2/1 in 1× PBS solution in rotating test tubes at 37° C. for 30 minutes. The single barcoded bead-cell complexes are purified from larger cell-bead complexes by filtration through a cell strainer (cell sieve with 40 or 100 micron pores) or by FACS (Becton Dickinson FACS Melody) based on forward and side scattering characteristics. FACS purification allows for separation of single barcoded bead-cell complexes from both multiple bead-cell complexes, empty bind beads and separate/not bind cells. Moreover, FACS allows for purification of only one or several specific cell type-barcoded bead complexes if barcoded beads have cell-type specific binding moiety (e.g. antibodies for CD8 for T cells, or CD19 for B cells, etc.).

### Example 4. Binding of Barcoded Beads and Cells on Solid Support

Single cell suspension of HEK293 cells (1×10⁶ cells, control and activated by TNF) transduced with barcoded lentiviral sgRNA library (80 sgRNAs targeting genes involved in NFkB signaling pathway) is bound to cell culture plastic dish (20-cm diameter) by incubation overnight in cell culture media (DMEM). In another protocol, the plastic surface is modified by spotting of micro patterning areas (e.g. 10-20 microns) separated from each other (e.g. 100-200 microns) of cell adhesion ligands (e.g. collagen, fibronectin, etc.) in a way that facilitates attachment of single cells in a spaced manner. The cells randomly attached to plastic are washed in 1× PBS and bound with Antibody-barcoded bead conjugate (beta2-microglobulin and CD293 antibody bead conjugates) comprising set of 180 reverse gene-specific primers specific for genes involved and regulated by NFkB signaling pathway. The barcoded antibody-bead conjugates are incubated with plastic-attached beads in plate shaker in 1× PBS for 30 minutes and cell-barcoded bead complexes attached to plastic surface are purified from non-attached beads by washing in 1× PBS buffer.

### Example 5. Hybridization of Barcoded Reverse Gene Specific Primers with Cellular Target Template RNAs

The example protocol below describes the methods for expression profiling of PBMC cells using barcoded beads with an immobilized set of reverse gene-specific primers targeting 1,200 cell marker specific genes. As a starting material, the protocol could employ any single cell suspension of interest in a1× PBS buffer at 1-10×10⁶ per ml. In addition, the protocol may use barcoded beads (e.g. 20 micron polystyrene beads) with covalently attached, e.g., through photocleavable linker, barcoded reverse gene specific primers designed for 1.2K cell marker genes, and non-covalently attached antibodies to universal cell surface antigens (e.g., anti-beta-2-microglobulin, anti-CD298) as described above. Furthermore, the protocol is based on the generation of cell-barcoded bead complexes though antibody-cell antigen binding, selection of the plurality of single cell-single barcoded bead complexes using FACS sorter and partitioning of produced cell-barcoded bead complexes in water-in-oil microdroplets comprising lysis/hybridization buffer using commercial BioRad ddSeq instruments and supporting reagents. The encapsulated cell-barcoded bead complexes are furthermore treated for limited cell lysis, release of RNA or DNA and hybridization of cellular RNA/DNA to barcoded reverse gene specific primers provided by bead.

Protocol for production of primed barcoded RNA templates from Normal Human PBMC sample:
1. Isolate PBMC from anticoagulated human blood sample using Ficoll-based density gradient centrifugation (e.g. SepMate tubes, StemCell Technologies)
2. Wash PBMC and resuspend in PBS at concentration of 1-10×10⁶ per ml.
3. Incubate PBMC with barcoded beads comprising attached cell binding antibodies (against beta2-microglobulin and CD293) at room temperature for 30 minutes
4. Analyze the cell suspension containing bead-cell complexes by flow cytometry to identify single cell-bead complexes by forward and side light scatter bivariate plot; and sort single cell-barcoded bead subset into 1× PBS. 01%BSA, 10% Ficoll400 aiming for a concentration of 150K cells per ml.
5. Partition the sorted barcoded bead-cell complexes (80 ul/12K cell-beads per chip) with 2× lysis/hybridization buffer (200 mM Tris-HCl, pH 7.6, 20 mM EDTA. 50 mM DTT, 1 M NaCl, 0.2% sacrosine) into microdroplets using ddSeq droplet generator instrument, oil-surfactant composition (EvaGreen) and manufacturer's protocol (BioRad). Transfer the generated droplet emulsion in 0.5-ml test tube.
6. Expose droplet emulsion (in test tube) to UV 365 light for 5 minutes to cleave photo-sensitive linker, thereby releasing barcoded reverse gene specific primers from beads.
7. Incubate the droplet emulsion in thermal cycler at 700 for 2 min, 50 C for 30 min to allow hybridization of cellular RNA (or if necessary DNA) released from cells by lysis with barcoded reverse gene specific primers released from the beads.
8. After hybridization step, remove the emulsion from thermal cycler, add 300 ul of 1× TCL buffer (Qiagen) and 40 ul of Droplet Disruptor (BioRad). After adding droplet disruptor, the emulsion will break down, forming oil (bottom) and water phase (upper layer).
9. Add 30 ul of magnetic oligo-dT beads (Thermo-Fisher) to water phase and shake the test tube at 1,200 rpm for 20 minutes.
10. Centrifuge the test tube to pellet magnetic oligo dT beads-barcoded reverse gene specific primer-RNA complexes (primed barcoded RNA template).
11. Wash the magnetic oligo dT beads-Rev GSP-RNA complexes three times with 1× TCW washing buffer (Qiagen) using magnetic stand.
12. Proceed to multiplex RT-PCR and NGS.

### Example 6. Multiplex RT-PCR Assay

### A. Design of Primers for Anchor Addition, First and Second PCR Steps

Design of Barcoded Forward and Barcoded Reverse gene specific primers with anchor1 (Fwd-anchor1-GSP primers) and anchor2 (Rev-anchor2-GSP primers) with 3′-extended suppression portions for primer extension steps and universal PCR primers (F-MP1GAC and R-MP2CAG) to amplify anchored cDNA fragments by PCR.

Sequences that are underlined are the common PCR suppression portions, and those in italics and bold are unique sequences for Fwd or Rev primers, respectively, and GSP is the gene-specific primer domain. The BC-Link is Barcode-Linker domain which comprises the composite barcode as described in more details above and could be present in only reverse (preferred embodiment), only in forward or in both reverse and forward primers.

```
              F-MP1GAC
AGCAGCACCGACCAGCAGAC
      AGCACCGACCAGCAGACA(BC-Link)FwdGSP>
          Fwd-Anc1-GSP cDNA Rev-Anc2-GSP
                                                                                  <RevGSP(Link-BC)AGACACGACCAGCCACGA
                                                                                                                  GACACGACCAGCCACGAGCA
                                                                                                                              R-MP2CAG
```

(from top to bottom SEQ ID NOs:25-28)
For simplicity the structures below show the design of primers and amplification products only for the preferred embodiment of using barcoded reverse and non-barcoded forward gene specific primer set:

```
              F-MP1GAC
AGCAGCACCGACCAGCAGAC
      AGCACCGACCAGCAGACA-FwdGSP>
          Fwd-Anc1-GSP cDNA Rev-Anc2-GSP
                                                                  <RevGSP(Link-BC)AGACACGACCAGCCACGA
                                                                                                                  GACACGACCAGCCACGAGCA
                                                                                                                              R-MP2CAG
```

(from top to bottom SEQ ID NOs:25, 20, 27 and 28)
In another embodiment, the forward gene specific primer set is replaced with template switching (TS) oligonucleotide comprising UMI sequence (e.g. VNVNVN) which allow to add anchor1 sequence during reverse transcription step. As discussed in more detail above, the TS oligonucleotide may have additional domains, e.g., a sample specific domain. The example design of primers is shown below:

```
              F-MP1GAC
AGCAGCACCGACCAGCAGAC
AGCAGCACCGACCAGCAGACAVNVNVNrGrGrG
        TS1 RNA
                                                                        <RevGSP(Link-BC)AGACACGACCAGCCACGA
                                                                                                          GACACGACCAGCCACGAGCA
                                                                                                                  R-MP2CAG
```

(from top to bottom SEQ ID NOs:25, 29, 27 and 28)
Wherein, N is dG, dC, dT and dA; V is dG, dC and dA; rGrGrG—three riboG nucleotide at the 3′-end of template switching oligonucleotide.

The resultant structure of amplified cDNA products after the two sequential primer extension steps using Barcoded Rev-anchor2-GSPs and Fwd-anchor1-GSPs and a first PCR step using universal F-MPIGAC and R-MP2CAG primers is shown below:

(60-250 nt)
(from left to right SEQ ID NOs: 30-31)
AGCAGCACCGACCAGCAGACA-FwdGSP-cDNA-RevGSP-Link-BC-TCTGTGCTGGTCGGTGCTCGT
(from left to right SEQ ID NOs: 32-33)
TCGTCGTGGCTGGTCGTCTGT-FwdGSP-cDNA-RevGSP-Link-BC-AGACACGACCAGCCACGAGCA

The resultant structure of amplified cDNA products after reverse transcription step and a first PCR step using universal F-MPIGAC and R-MP2CAG primers is shown below:

(60-250 nt)
(from left to right SEQ ID NOs: 34-35)
AGCAGCACCGACCAGCAGACA-G(2-5)-cDNA-RevGSP-Link-BC-TCTGTGCTGGTCGGTGCTCGT
(from left to right SEQ ID NOs: 36-37)
TCGTCGTGGCTGGTCGTCTGT-C(2-5)-cDNA-RevGSP-Link-BC-AGACACGACCAGCCACGAGCA

The amplified after first PCR cDNA products are then subjected to a second round of PCR to add Illumina P7, P5 sequencing adaptors. PCR primers for the second PCR step comprise anchor 1 and anchor 2 binding domains, indexing (underlined) domains (optional domains, can be used if experiment requires to combine the different samples together for NGS step) and P5 or P7 sequences necessary for cluster formation in Illumina NGS instrument, as illustrated below:

Set of Forward Indexing Primers for 2^{nd} PCR step:
FP7-A1Ind-A
(SEQ ID NO: 38)
AGCAGAAGACGGCATACCAGATATACGACAGCAGCAGCACCGACCAGCAGACA
FP7-A1Ind-B
(SEQ ID NO: 39)
AGCAGAAGACGGCATACCAGATACTGATGAGCAGCAGCACCGACCAGCAGACA
FP7-A1Ind-C
(SEQ ID NO: 40)
AGCAGAAGACGGCATACCAGATAGCATCAAGCAGCAGCACCGACCAGCAGACA
FP7-A1Ind-D
(SEQ ID NO: 41)
AGCAGAAGACGGCATACCAGATAAGTCGTAGCAGCAGCACCGACCAGCAGACA
FP7-A1Ind-E
(SEQ ID NO: 42)
AGCAGAAGACGGCATACGAGATATCGCATAGCAGCAGCACCGACCAGCAGACA
FP7-A1Ind-F
(SEQ ID NO: 43)
AGCAGAAGACGGCATACGAGATACATAGCAGCAGCAGCACCGACCAGCAGACA
Set of Reverse Indexing Primers for 2^{nd} PCR step:
RP5-A2Ind-A
(SEQ ID NO: 44)
ACGGCGACCACCGAGATCTACACATACGACACGACGAGCACCGACCAGCACAGA
RP5-A2Ind-B
(SEQ ID NO: 45)
ACGGCGACCACCGAGATCTACACACTGATGACGACGAGCACCGACCAGCACAGA
RP5-A2Ind-C
(SEQ ID NO: 46)
ACGGCGACCACCGAGATCTACACAGCATCAACGACGAGCACCGACCAGCACAGA
RP5-A2Ind-D
(SEQ ID NO: 47)
ACGGCGACCACCGAGATCTACACAAGTCGTACGACGAGCACCGACCAGCACAGA
RP5-A2Ind-E
(SEQ ID NO: 48)
ACGGCGACCACCGAGATCTACACATCGCATACGACGAGCACCGACCAGCACAGA
RP5-A2Ind-E
(SEQ ID NO: 49)
ACGGCGACCACCGAGATCTACACACATAGCACGACGAGCACCGACCAGCACAGA
Set of Forward and Reverse Non-indexing Primers for 2^{nd} PCR step:
FP7-A1
(SEQ ID NO: 50)
AGCAGAAGACGGCATACGAGATAGCAGCAGCACCGACCAGCAGACA
RP5-A2
(SEQ ID NO: 51)
ACGGCGACCACCGAGATCTACACACGACGAGCACCGACCAGCACAGA

After a second PCR step with Forward and Reverse indexing primers the final amplicon structure, flanked with P7 and P5 Illumina's adaptor sequences, and ready for NGS step is shown below: (SEQ ID DOS: 53-54)

(from left to right SEQ ID NOs: 52-53)
P7(ind)AGCAGCACCGACCAGCAGACA-FwdGSP-cDNA-RevGSP(LinkBC)TCTGTGCTGGTCGGTGCTCGT(Ind)P5
(from left to right SEQ ID NOs: 54-55)
P7(Ind)TCGTCGTGGCTGGTCGTCTGT-FwdGSP-cDNA-RevGSP(LinkBC)AGACACGACCAGCCACGAGCA(Ind)P5

The sequences of primers for NGS sequencing (e.g. Illumina NextSeq500 platform) of cDNA inserts, barcode domain and indexes are provided below:

SeqDNAlink-Rev
(SEQ ID NO: 56)
TGGCGTGCTGGCGGTGCTGGTCGGT
SeqDNA-Fwd
(SEQ ID NO 57)
AGCAGCAGCACCGACCAGCAGACA
SeqBarcode-Fwd
(SEQ ID NO: 58)
ACCGACCAGCACCGCCAGCACGCCA
Optional sequencing primers:
SeqIND-Fwd
(SEQ ID NO: 59)
TCTGTGCTGGTCGGTGCTCGTCGT
SeqIND-Rev
(SEQ ID NO: 60)
TGTCTGCTGGTCGGTGCTGCTGCT
SeqDNA-Rev
(SEQ ID NO: 61)
ACGACGAGCACCGACCAGCACAGA

Example of program for NGS sequencing of amplified barcoded cDNA products in Next Seq500 machine using 150-nt sequencing kit is shown below:
Read 1: SeqDNAlink-Rev>81 cycles
Ind 1: SeqIND-Rev>6 cycles
Ind 2: SeqBarcode-Fwd>38 cycles
Read 2: SeqDNA-Fwd>35 cycles
   The read number for SeqBarcode-Fwd primer could depend of the design of specific barcode domain cassette. The number of read 38 is selected for reading complex sample barcode domain with the structure: Antibody barcode(6)-Sample barcode(6)-Bead barcode(14)-UMI(12).
   B. Protocol for Multiplex AT-PCR Amplification of Target Genes for Expression Profiling or Mutation Analysis Starting from Primed Barcoded RNA Template (Example 5).

Step 1. Production of barcoded cDNA product. Magnetic oligo dT beads-barcoded Rev GSP-RNA complexes isolated from 10,000 of PBMC cells (example 5) incubated in 20 ul of reaction mix comprising 1× GC buffer, dNTP (500 uM) and 20 units of Exonuclease I (New England BioLabs) at 37° C. for 20 min. The cDNA synthesis is initiated by adding to the reaction mix comprising buffer and dNTP of 200 units of Maxima Reverse Transcriptase (Thermo-Fisher) and incubation at 56° C. for 30 min, followed by reverse transcriptase inactivation step at 85° C. for 5 min. In the case of using template switching (TS) oligonucleotide, the TS oligonucleotide is added in reverse transcriptase reaction mix at 1-3 μM concentration and incubated at 42° C. for 30 min, followed by incubation at 85° C. for 5 min. Step 2 is omitted in the case of using TS oligonucleotide as universal anchor primer binding site is incorporated in barcoded extension product during reverse transcription step.

Step 2. Forward primer extension. Barcoded cDNA is primed (add universal anchors 1) using mix of Forward-anchor1-GSP primers (5 nM final concentration for each primer) in 40-ul reaction mix comprising 1× GC buffer, dNTP (250 uM) and Phusion II (4 units, Thermo-Fisher) for 1 cycles at (98° C. for 1 min, 64° C. for 30 min). Non-extended forward primers are removed by adding to the reaction mix 20 units of Exonuclease I (New England Biolabs) and incubation at 37° C. for 30 min, 95° C. for 5 min.

Step 3. 1^{st} PCR step. Whole volume (40-μl) of barcoded anchored cDNA fragments (from Step 2) are amplified in 80-μl reaction mix comprising 1× GC Buffer, dNTP (200 uM), universal PCR primers F-MP1GAC and R-MP2CAG and Phusion II DNA polymerase (20 units, Thermo-Fisher) for 16 cycles at 98° C. for 10 sec, 72° C. for 20 sec.

Step 4. 2^{nd} PCR step. 5-μl aliquot of 1st PCR is amplified in 100-μl of PCR mix comprising 1× GC Buffer, dNTP (200 μM), indexed (specific for the each of several samples) or non-indexed (only for one sample) Fwd and Rev PCR primers and Phusion II (20 units, Thermo-Fisher) for 7 cycles at 98° C. for 10 sec, 72° C. for 20 sec.

Step 5. The amplified PCR products are analyzed in 3.5% agarose-1× TAE gel to optimize the cycle number and finally digested with exonuclease I (20 units, New England Biolabs), by incubating the reaction mix and 37° C. for 30 min, and purified by AMPure magnetic beads (1.5× volume, Beckman-Coulter) using manufacturer's protocol. Purified PCR products are quantitated by Qubit (Thermo-Fisher) and if necessary different samples are mixed together (at equal amount), diluted to 10 nM and sequenced in NextSeq500 using Illumina paired-end protocol and reagents for 150 cycles.

### C. Protocol for Multiplex RT-PCR Amplification of Target Genes for Expression Profiling or Mutation Analysis in Single Cells Using Barcoded Reverse Gene Specific Primer Set in Microwells.

Step 1. Individual cells (5,000-10,000) are deposited by FACS in a separate wells together with barcoded reverse gene specific primer set immobilized on bead through photocleavable linker (ChemB-T25-PCI-Anc2-BC-L2, ChemGenes, see structure above) in 1× TCL lysis-hybridization buffer (Qiagen).

Step 2. Barcoded reverse gene specific primers are released from beads by UV365 nm treatment (20 watts) for 5 minutes and hybridized with target RNA templates (present in lysates in a separate compartments) at 60° C. for 30 min. The hybridized complexes between target RNA and barcoded reverse gene specific primers are combined together and purified using binding to oligo dT25-beads by washing the beads three times in 1×SSC buffer. The purified target RNA-Barcoded reverse gene specific primer complexes are treated with thermosensitive exonuclease I (20 units, New England BioLabs) in 20-μl of 1× GC buffer at 37° C. for 30-min, 50° C. for 5 min. In an alternative protocol, the hybridized complexes between target RNA and barcoded reverse gene specific primers are combined together, purified using RNA/DNA micro kit (Qiagen) and treated with thermosensitive exonuclease I.

Step 3. Reverse primer extension step. RNA is converted to barcoded cDNA from barcoded reverse gene specific primers (hybridized to target RNA in Step 2) in 40-μl of reaction mix comprising 1× GC buffer, dNTP (500 uM), ThermaStop-RT (80 units, ThermaGenix) and Maxima Reverse Transcriptase (400 units, Thermo-Fisher) at 55° C. for 30 min. In the case of using template switching (TS) oligonucleotide, the TS oligonucleotide is added in reverse transcriptase reaction mix at 1-3 μM concentration and incubated at 42° C. for 30 min, followed by incubation at 85° C. for 5 min. Step 2 is omitted in the case of using TS oligonucleotide as universal anchor primer binding site is incorporated in barcoded extension product during reverse transcription step.

Step 4. Forward primer extension step. Barcoded cDNA (generated in Step 3) is primed using mix of Forward-anchor1-GSP primers (5 nM final concentration for the each primer) in 50-ul reaction mix comprising 1× GC buffer, dNTP (250 uM) and Phusion II (10 units, Thermo-Fisher) for 1 cycles at (98° C. for 1 min, 64° C. for 30 min) and treated with exonuclease I (20 units) at 37° C. for 30-min.

Step 5. 1^{st} PCR step. Whole volume (50-μl) of barcoded anchored cDNA fragments (from Step 4) are amplified in 100-μl reaction mix comprising 1× GC Buffer, dNTP (200 uM), universal PCR primers F-MP1GAC and R-MP2CAG and Phusion II (20 units, Thermo-Fisher) for 14 cycles at (98° C. for 10 sec, 72° C. for 20 sec).

Step 6. 2^{nd} PCR step. 5-μl aliquot of 1st PCR is amplified in 100-μl of PCR mix comprising 1× HF Buffer, dNTP (200 μM), indexed (specific for the each of several samples) or non-indexed (only for one sample) Fwd and Rev PCR primers and Phusion II (20 units, Thermo-Fisher) for 7 cycles at (98° C. for 10 sec, 72° C. for 20 sec).

Step 7. The amplified PCR products are analyzed in 3.5% agarose-1× TAE gel to optimize the cycle number and finally digested with exonuclease I (20 units, New England Biolabs), incubated and 37° C. for 30 min, inactivated at 65° C. for 15 min and purified by AMPure beads (1.5× volume) using manufacturer's protocol (Beckman-Coulter). Purified PCR products were quantitated by Qubit (Thermo-fisher) and if necessary different samples were mixed together (at equal amount), diluted to 10 nM and sequenced in NextSeq500 using IIlumina paired-end protocol and reagents for 150 cycles.

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended hi the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended hi the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

Although the foregoing invention has been described hi some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the is inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. Moreover, nothing disclosed herein is intended to be dedicated to the public regardless of whether such disclosure is explicitly recited in the claims.

The scope of the present invention, therefore, is not intended to be limited to the exemplary embodiments shown and described herein. Rather, the scope and spirit of present invention is embodied by the appended claims. In the claims, 35 U.S.C. § 112(f) or 35 U.S.C. § 112(6) is expressly defined as being invoked for a limitation in the claim only when the exact phrase “means for” or the exact phrase “step for” is recited at the beginning of such limitation in the claim; if such exact phrase is not used in a limitation in the claim, then 35 U.S.C. § 112 (f) or 35 U.S.C. § 112(6) is not invoked.

1. A method of preparing barcoded nucleic acids, the method comprising:
producing a plurality of partitioned cell/barcoded bead complexes from:
a cellular sample; and
a plurality of distinct barcoded beads comprising a plurality barcoded reverse gene-specific primers;
hybridizing gene-specific template binding domains of barcoded reverse gene-specific primers of the beads to template nucleic acids of the cells of the partitioned separated cell/barcoded bead complexes to produce primed template nucleic acids; and
subjecting the primed template nucleic acids to primer extension reaction conditions sufficient to produce barcoded nucleic acids.

2. The method according to claim 1, wherein the cell/barcoded bead complexes are partitioned in aqueous droplets present in an immiscible liquid.

3. The method according to claim 1, wherein the cell/barcoded bead complexes are partitioned into microwells.

4. The method according to according to claim 1, wherein the primer extension reaction employs a template switching oligonucleotide.

5. The method according to according to claim 1, wherein the cell/barcoded bead complexes comprise complexes made up of a single cell or component thereof and a single bead.

6. The method according to claim 5, wherein the cell/barcoded bead compositions comprise complexes of a cell nucleus and a single bead.

7. The method according to according to claim 1, wherein the method comprises releasing the barcoded reverse gene-specific primers from the barcoded beads prior to hybridizing gene-specific template binding domains of barcoded reverse gene-specific primers to template nucleic acids of the cells to produce primed template nucleic acids.

8. The method according to claim 7, wherein the barcoded reverse gene-specific primers are bound to the beads by a cleavable linker.

9. The method according to according to claim 1, wherein the method comprises lysing cells of cell-barcoded bead complexes prior to hybridizing template binding domains of barcoded reverse gene-specific primers to template nucleic acids of the cells to produce primed template nucleic acids.

10. The method according to according to claim 1, wherein the barcoded beads comprise a cellular binding moiety.

11-12. (canceled)

13. The method according to according to claim 1, wherein the barcoded beads comprise 100 or more distinct gene-specific barcoded reverse primers.

14. The method according to according to claim 1, wherein the gene-specific template binding domains are experimentally validated.

15. The method according to according to claim 1, wherein the barcoded reverse gene-specific primers further comprise an anchor domain.

16. The method according to according to claim 1, wherein the barcoded reverse gene-specific primers further comprise a unique molecular identifier (UMI) domain.

17. The method according to according to claim 1, wherein the method further comprises amplifying the barcoded nucleic acids to produce an amplified nucleic acid composition.

18. The method according to claim 17, wherein the amplifying comprises primer extension from a plurality of forward gene-specific primers that comprise an anchor domain and a template binding domain complementary to the barcoded nucleic acids.

19. The method according to according to claim 1, wherein the method further comprises sequencing the amplified nucleic acid composition.

20. The method according to claim 19, wherein the sequence is performed by a NGS protocol.

21. The method according to according to claim 1, wherein the method further comprises pooling.

22-41. (canceled)


SEQUENCE LISTING (61 sequences)

SEQ ID NO: 1 - DNA, 18 bp, Artificial sequence
  synthetic sequence
agcaccgaccagcagaca

SEQ ID NO: 2 - DNA, 18 bp, Artificial sequence
  synthetic sequence
agcaccgaccagcacaga

SEQ ID NO: 3 - DNA, 20 bp, Artificial sequence
  synthetic sequence
aatgatacggcgaccaccga

SEQ ID NO: 4 - DNA, 24 bp, Artificial sequence
  synthetic sequence
caagcagaagacggcatacgagat

SEQ ID NO: 5 - DNA, 33 bp, Artificial sequence
  synthetic sequence
acactctttccctacacgacgctcttccgatct

SEQ ID NO: 6 - DNA, 34 bp, Artificial sequence
  synthetic sequence
gtgactggagttcagacgtgtgctcttccgatct

SEQ ID NO: 7 - DNA, 30 bp, Artificial sequence
  synthetic sequence
ccatctcatccctgcgtgtctccgactcag

SEQ ID NO: 8 - DNA, 23 bp, Artificial sequence
  synthetic sequence
cctctctatgggcagtcggtgat

SEQ ID NO: 9 - DNA, 21 bp, Artificial sequence
  synthetic sequence
  misc_feature (1)..(1): The nucleotide at position 1 is associated with a linker, e.g., a cleavable linker
  misc_feature (21)..(21): The nucleotide at position 21 is associated with a barcode sequence
acgagcaccgaccagcacaga

SEQ ID NO: 10 - DNA, 12 bp, Artificial sequence
  synthetic sequence
  misc_feature (1)..(1): The nucleotide at position 1 is associated with a barcode sequence
accgcacgaccg

SEQ ID NO: 11 - DNA, 13 bp, Artificial sequence
  synthetic sequence
  misc_feature (13)..(13): The nucleotide at position 13 is associated with a reverse gene specific primer set (RevGSP)
ccacgaccagcca

SEQ ID NO: 12 - DNA, 26 bp, Artificial sequence
  synthetic sequence
atggctggtcgtggcggtcgtgcggt

SEQ ID NO: 13 - DNA, 41 bp, Artificial sequence
  synthetic sequence
acgagcaccgaccagcacagagaacaaacaccgcacgaccg

SEQ ID NO: 14 - DNA, 41 bp, Artificial sequence
  synthetic sequence
acgagcaccgaccagcacagaggcgaaacaccgcacgaccg

SEQ ID NO: 15 - DNA, 41 bp, Artificial sequence
  synthetic sequence
acgagcaccgaccagcacagagcaaaaggaccgcacgaccg

SEQ ID NO: 16 - DNA, 44 bp, Artificial sequence
  synthetic sequence
  misc_feature (1)..(1): The nucleotide at position 1 is associated with a photocleavable linker (PClinker)
  misc_feature (21)..(21): n is a, c, g, or t
  misc_feature (24)..(24): n is a, c, g, or t
agcaccgaccagcacagavvnvvnvvcatcagaccgcacgaccg

SEQ ID NO: 17 - DNA, 45 bp, Artificial sequence
  synthetic sequence
  misc_feature (1)..(1): The nucleotide at position 1 is associated with a bi-sulfite linker (SSlinker)
  misc_feature (21)..(21): n is a, c, g, or t
  misc_feature (24)..(24): n is a, c, g, or t
agcaccgaccagcacagavvnvvnvvcagcatgaccgcacgaccg

SEQ ID NO: 18 - DNA, 25 bp, Artificial sequence
  synthetic sequence
  misc_feature (1)..(1): The nucleotide at position 1 is associated with a universal molecular identifier (UMI) Barcode
  misc_feature (25)..(25): The nucleotide at position 25 is associated with a reverse gene specific primer set (RevGSP)
accgcacgaccgccacgaccagcca

SEQ ID NO: 19 - DNA, 18 bp, Artificial sequence
  synthetic sequence
  misc_feature (18)..(18): The nucleotide at position 18 is associated with a universal molecular identifier (UMI) barcode
agcaccgaccagcagaca

SEQ ID NO: 20 - DNA, 18 bp, Artificial sequence
  synthetic sequence
  misc_feature (18)..(18): The nucleotide at position 18 is associated with a foward gene specific primer set (FwdGSP)
agcaccgaccagcagaca

SEQ ID NO: 21 - DNA, 13 bp, Artificial sequence
  synthetic sequence
  misc_feature (13)..(13): The nucleotide at position 13 is associated with a moiety, where the moiety may include lipids (cholesterol, fatty acid, like stearoylic or palmitic acid), and oligonucleotide aptamers, e.g. CD4 aptamer
ccacgaccagcca

SEQ ID NO: 22 - DNA, 45 bp, Artificial sequence
  synthetic sequence
ccaccaccgtacaattcgctttcttttttcattacctactctggc

SEQ ID NO: 23 - DNA, 13 bp, Artificial sequence
  synthetic sequence
  misc_feature (13)..(13): The nucleotide at position 13 is amino-modified
ccacgaccagcca

SEQ ID NO: 24 - DNA, 25 bp, Artificial sequence
  synthetic sequence
  misc_feature (1)..(1): The nucleotide at position 1 is amino-modified
tggctggtcgtggcggtcgtgcggt

SEQ ID NO: 25 - DNA, 20 bp, Artificial sequence
  synthetic sequence
agcagcaccgaccagcagac

SEQ ID NO: 26 - DNA, 18 bp, Artificial sequence
  synthetic sequence
  misc_feature (18)..(18): The nucleotide at position 18 is associated with (BC-Link)FwdGSP, where BC-Link is Barcode-Linker domain and FwdGSP is a forward gene specific primer domain
agcaccgaccagcagaca

SEQ ID NO: 27 - DNA, 18 bp, Artificial sequence
  synthetic sequence
  misc_feature (1)..(1): The nucleotide at position 1 is associated with (BC-Link)RevGSP, where BC-Link is Barcode-Linker domain and RevGSP is a reverse gene specific primer domain
agacacgaccagccacga

SEQ ID NO: 28 - DNA, 20 bp, Artificial sequence
  synthetic sequence
gacacgaccagccacgagca

SEQ ID NO: 29 - DNA, 30 bp, Artificial sequence
  synthetic sequence
  misc_feature (22)..(22): V is selected from dG, dC and dA
  misc_feature (23)..(23): N is selected from dG, dC, dT and dA
  misc_feature (24)..(24): V is selected from dG, dC and dA
  misc_feature (25)..(25): N is selected from dG, dC, dT and dA
  misc_feature (26)..(26): V is selected from dG, dC and dA
  misc_feature (27)..(27): N is selected from dG, dC, dT and dA
  misc_feature (28)..(30): The nucleotides at positions 28-30 comprise riboG
agcagcaccgaccagcagacavnvnvnggg

SEQ ID NO: 30 - DNA, 21 bp, Artificial sequence
  synthetic sequence
  misc_feature (21)..(21): The nucleotide at position 21 is associated with a forward gene specific primer set (fwdGSP)
agcagcaccgaccagcagaca

SEQ ID NO: 31 - DNA, 21 bp, Artificial sequence
  synthetic sequence
  misc_feature (1)..(1): The nucleotide at position 1 is associated with (BC-Link)RevGSP, where BC-Link is Barcode-Linker domain and RevGSP is a reverse gene specific primer domain
tctgtgctggtcggtgctcgt

SEQ ID NO: 32 - DNA, 21 bp, Artificial sequence
  synthetic sequence
  misc_feature (21)..(21): The nucleotide at position 21 is associated with a forward gene specific primer set (fwdGSP)
tcgtcgtggctggtcgtctgt

SEQ ID NO: 33 - DNA, 21 bp, Artificial sequence
  synthetic sequence
  misc_feature (1)..(1): The nucleotide at position 1 is associated with (BC-Link)RevGSP, where BC-Link is Barcode-Linker domain and RevGSP is a reverse gene specific primer domain
agacacgaccagccacgagca

SEQ ID NO: 34 - DNA, 21 bp, Artificial sequence
  synthetic sequence
  misc_feature (21)..(21): The nucleotide at position 21 is associated with (G)n, where n is an integer ranging from 2 to 5
agcagcaccgaccagcagaca

SEQ ID NO: 35 - DNA, 21 bp, Artificial sequence
  synthetic sequence
  misc_feature (1)..(1): The nucleotide at position 1 is associated with (BC-Link)RevGSP, where BC-Link is Barcode-Linker domain and RevGSP is a reverse gene specific primer domain
tctgtgctggtcggtgctcgt

SEQ ID NO: 36 - DNA, 21 bp, Artificial sequence
  synthetic sequence
  misc_feature (21)..(21): The nucleotide at position 21 is associated with (C)n, where n is an integer ranging from 2 to 5
tcgtcgtggctggtcgtctgt

SEQ ID NO: 37 - DNA, 21 bp, Artificial sequence
  synthetic sequence
  misc_feature (1)..(1): The nucleotide at position 1 is associated with (BC-Link)RevGSP, where BC-Link is Barcode-Linker domain and RevGSP is a reverse gene specific primer domain
agacacgaccagccacgagca

SEQ ID NO: 38 - DNA, 53 bp, Artificial sequence
  synthetic sequence
agcagaagacggcatacgagatatacgacagcagcagcaccgaccagcagaca

SEQ ID NO: 39 - DNA, 53 bp, Artificial sequence
  synthetic sequence
agcagaagacggcatacgagatactgatgagcagcagcaccgaccagcagaca

SEQ ID NO: 40 - DNA, 53 bp, Artificial sequence
  synthetic sequence
agcagaagacggcatacgagatagcatcaagcagcagcaccgaccagcagaca

SEQ ID NO: 41 - DNA, 53 bp, Artificial sequence
  synthetic sequence
agcagaagacggcatacgagataagtcgtagcagcagcaccgaccagcagaca

SEQ ID NO: 42 - DNA, 53 bp, Artificial sequence
  synthetic sequence
agcagaagacggcatacgagatatcgcatagcagcagcaccgaccagcagaca

SEQ ID NO: 43 - DNA, 53 bp, Artificial sequence
  synthetic sequence
agcagaagacggcatacgagatacatagcagcagcagcaccgaccagcagaca

SEQ ID NO: 44 - DNA, 54 bp, Artificial sequence
  synthetic sequence
acggcgaccaccgagatctacacatacgacacgacgagcaccgaccagcacaga

SEQ ID NO: 45 - DNA, 54 bp, Artificial sequence
  synthetic sequence
acggcgaccaccgagatctacacactgatgacgacgagcaccgaccagcacaga

SEQ ID NO: 46 - DNA, 54 bp, Artificial sequence
  synthetic sequence
acggcgaccaccgagatctacacagcatcaacgacgagcaccgaccagcacaga

SEQ ID NO: 47 - DNA, 54 bp, Artificial sequence
  synthetic sequence
acggcgaccaccgagatctacacaagtcgtacgacgagcaccgaccagcacaga

SEQ ID NO: 48 - DNA, 54 bp, Artificial sequence
  synthetic sequence
acggcgaccaccgagatctacacatcgcatacgacgagcaccgaccagcacaga

SEQ ID NO: 49 - DNA, 54 bp, Artificial sequence
  synthetic sequence
acggcgaccaccgagatctacacacatagcacgacgagcaccgaccagcacaga

SEQ ID NO: 50 - DNA, 46 bp, Artificial sequence
  synthetic sequence
agcagaagacggcatacgagatagcagcagcaccgaccagcagaca

SEQ ID NO: 51 - DNA, 47 bp, Artificial sequence
  synthetic sequence
acggcgaccaccgagatctacacacgacgagcaccgaccagcacaga

SEQ ID NO: 52 - DNA, 21 bp, Artificial sequence
  synthetic sequence
  misc_feature (1)..(1): The nucleotide at position 1 is associated with a P7 Illumina adaptor sequence
  misc_feature (21)..(21): The nucleotide at position 21 is associated with a forward gene specific primer set (fwdGSP)
agcagcaccgaccagcagaca

SEQ ID NO: 53 - DNA, 21 bp, Artificial sequence
  synthetic sequence
  misc_feature (1)..(1): The nucleotide at position 1 is associated with (BC-Link)RevGSP, where BC-Link is Barcode-Linker domain and RevGSP is a reverse gene specific primer domain
  misc_feature (21)..(21): The nucleotide at position 21 is associated with a P5 Illumina adaptor sequence
tctgtgctggtcggtgctcgt

SEQ ID NO: 54 - DNA, 21 bp, Artificial sequence
  synthetic sequence
  misc_feature (1)..(1): The nucleotide at position 1 is associated with a P7 Illumina adaptor sequence
  misc_feature (21)..(21): The nucleotide at position 21 is associated with a forward gene specific primer set (fwdGSP)
tcgtcgtggctggtcgtctgt

SEQ ID NO: 55 - DNA, 21 bp, Artificial sequence
  synthetic sequence
  misc_feature (1)..(1): The nucleotide at position 1 is associated with (BC-Link)RevGSP, where BC-Link is Barcode-Linker domain and RevGSP is a reverse gene specific primer domain
  misc_feature (21)..(21): The nucleotide at position 21 is associated with a P5 Illumina adaptor sequence
agacacgaccagccacgagca

SEQ ID NO: 56 - DNA, 25 bp, Artificial sequence
  synthetic sequence
tggcgtgctggcggtgctggtcggt

SEQ ID NO: 57 - DNA, 24 bp, Artificial sequence
  synthetic sequence
agcagcagcaccgaccagcagaca

SEQ ID NO: 58 - DNA, 25 bp, Artificial sequence
  synthetic sequence
accgaccagcaccgccagcacgcca

SEQ ID NO: 59 - DNA, 24 bp, Artificial sequence
  synthetic sequence
tctgtgctggtcggtgctcgtcgt

SEQ ID NO: 60 - DNA, 24 bp, Artificial sequence
  synthetic sequence
tgtctgctggtcggtgctgctgct

SEQ ID NO: 61 - DNA, 24 bp, Artificial sequence
  synthetic sequence
acgacgagcaccgaccagcacaga